SEQUENCE LISTING (23 sequences)

SEQ ID NO: 1 - protein, 346 aa, Trypanosoma cruzi
MAPAAVDWRARGAVTAVKDQGQCGSCWAFSAIGNVECQWFLAGHPLTNLSEQMLVSCDKTDSGCSGGLMNNAFEWIVQENNGAVYTEDSYPYASGEGISPPCTTSGHTVGATITGHVELPQDEAQIAAWLAVNGPVAVAVDASSWMTYTGGVMTSCVSEQLDHGVLLVGYNDSAAVPYWIIKNSWTTQWGEEGYIRIAKGSNQCLVKEEASSAVVGGPGPTPEPTTTTTTSAPGPSPSYFVQMSCTDAACIVGCENVTLPTGQCLLTTSGVSAIVTCGAETLTEEVFLTSTHCSGPSVRSSVPLNKCNRLLRGSVEFFCGSSSSGRLADVDRQRRHQPYHSRHRRL

SEQ ID NO: 2 - protein, 216 aa, Trypanosoma cruzi
MAPAAVDWRARGAVTAVKDQGQCGSCWAFSAIGNVECQWFLAGHPLTNLSEQMLVSCDKTDSGCSGGLMNNAFEWIVQENNGAVYTEDSYPYASGEGISPPCTTSGHTVGATITGHVELPQDEAQIAAWLAVNGPVAVAVDASSWMTYTGGVMTSCVSEQLDHGVLLVGYNDSAAVPYWIIKNSWTTQWGEEGYIRIAKGSNQCLVKEEASSAVVG

SEQ ID NO: 3 - protein, 346 aa, Artificial Sequence
  engineered cruzipain T. cruzi without labels
MAPAAVDWRARGAVTAVKDQGQCGSGWAFSAIGNVECQWFLAGHPLTNLSEQMLVSCDKTDSGCSSGLMNNAFEWIVQENNGAVYTEDSYPYASGEGISPPCTTSGHTVGATITGHVELPQDEAQIAAWLAVNGPVAVCVDASSWMTYTGGVMTSCVSEQLDHGVLLVGYNDSAAVPYWIIKNSWTTQWGEEGYIRIAKGSNQCLVKEHASSAVVGGPGPTPEPTTTTTTSAPGPSPSYFVQMSCTDAACIVGCENVTLPTGQCLLTTSGVSAIVTCGAETLTEEVFLTSTHCSGPSVRSSVPLNKCNRLLRGSVEFFCGSSSSGRLADVDRQRRHQPYHSRHRRL

SEQ ID NO: 4 - protein, 216 aa, Artificial Sequence
  engineered cruzain T. cruzi without labels
MAPAAVDWRARGAVTAVKDQGQCGSGWAFSAIGNVECQWFLAGHPLTNLSEQMLVSCDKTDSGCSSGLMNNAFEWIVQENNGAVYTEDSYPYASGEGISPPCTTSGHTVGATITGHVELPQDEAQIAAWLAVNGPVAVCVDASSWMTYTGGVMTSCVSEQLDHGVLLVGYNDSAAVPYWIIKNSWTTQWGEEGYIRIAKGSNQCLVKEHASSAVVG

SEQ ID NO: 5 - protein, 353 aa, Artificial Sequence
  engineered cruzipain T. cruzi with labels
MCAPAAVDWRARGAVTAVKDQGQCGSGWAFSAIGNVECQWFLAGHPLTNLSEQMLVSCDKTDSGCSSGLMNNAFEWIVQENNGAVYTEDSYPYASGEGISPPCTTSGHTVGATITGHVELPQDEAQIAAWLAVNGPVAVCVDASSWMTYTGGVMTSCVSEQLDHGVLLVGYNDSAAVPYWIIKNSWTTQWGEEGYIRIAKGSNQCLVKEHASSAVVGGPGPTPEPTTTTTTSAPGPSPSYFVQMSCTDAACIVGCENVTLPTGQCLLTTSGVSAIVTCGAETLTEEVFLTSTHCSGPSVRSSVPLNKCNRLLRGSVEFFCGSSSSGRLADVDRQRRHQPYHSRHRRLHHHHHH

SEQ ID NO: 6 - protein, 223 aa, Artificial Sequence
  engineered cruzipain T. cruzi with labels
MCAPAAVDWRARGAVTAVKDQGQCGSGWAFSAIGNVECQWFLAGHPLTNLSEQMLVSCDKTDSGCSSGLMNNAFEWIVQENNGAVYTEDSYPYASGEGISPPCTTSGHTVGATITGHVELPQDEAQIAAWLAVNGPVAVCVDASSWMTYTGGVMTSCVSEQLDHGVLLVGYNDSAAVPYWIIKNSWTTQWGEEGYIRIAKGSNQCLVKEHASSAVVGHHHHHH

SEQ ID NO: 7 - protein, 408 aa, Thermus aquaticus
MDAFTENLNKLAELAIRVGLNLEEGQEIVATAPIEAVDFVRLLAEKAYENGASLFTVLYGDNLIARKRLALVPEAHLDRAPAWLYEGMAKAFHEGAARLAVSGNDPKALEGLPPERVGRAQQAQSRAYRPTLSAITEFVTNWTIVPFAHPGWAKAVFPGLPEEEAVQRLWQAIFQATRVDQEDPVAAWEAHNRVLHAKVAFLNEKRFHALHFQGPGTDLTVGLAEGHLWQGGATPTKKGRLCNPNLPTEEVFTAPHRERVEGVVRASRPLALSGQLVEGLWARFEGGVAVEVGAEKGEEVLKKLLDTDEGARRLGEVALVPADNPIAKTGLVFFDTLFDENAASHIAFGQAYAENLEGRPSGEEFRRRGGNESMVHVDWMIGSEEVDVDGLLEDGTRVPLMRRGRWVI

SEQ ID NO: 8 - protein, 415 aa, Artificial Sequence
  labeled TaqAPT
MCDAFTENLNKLAELAIRVGLNLEEGQEIVATAPIEAVDFVRLLAEKAYENGASLFTVLYGDNLIARKRLALVPEAHLDRAPAWLYEGMAKAFHEGAARLAVSGNDPKALEGLPPERVGRAQQAQSRAYRPTLSAITEFVTNWTIVPFAHPGWAKAVFPGLPEEEAVQRLWQAIFQATRVDQEDPVAAWEAHNRVLHAKVAFLNEKRFHALHFQGPGTDLTVGLAEGHLWQGGATPTKKGRLCNPNLPTEEVFTAPHRERVEGVVRASRPLALSGQLVEGLWARFEGGVAVEVGAEKGEEVLKKLLDTDEGARRLGEVALVPADNPIAKTGLVFFDTLFDENAASHIAFGQAYAENLEGRPSGEEFRRRGGNESMVHVDWMIGSEEVDVDGLLEDGTRVPLMRRGRWVIHHHHHH

SEQ ID NO: 9 - protein, 285 aa, Streptomyces griseus
MAPDIPLANVKAHLTQLSTIAANNGGNRAHGRPGYKASVDYVKAKLDAAGYTTTLQQFTSGGATGYNLIADWPGGDPNKVLMAGAHLDSVSSGAGINDNGSGSAAVLETALAVSRAGYQPDKHLRFAWWGAEELGLIGSKYYVNNLPSADRSKLAGYLNFDMIGSPNPGYFVYDDDPVIEKTFKDYFAGLNVPTEIETEGDGRSDHAPFKNVGVPVGGLFTGAGYTKSAAQAQKWGGTAGQAFDRCYHSSCDSLSNINDTALDRNSDAAAHAIWTLSSGTGEPPT

SEQ ID NO: 10 - protein, 299 aa, Aeromonas proteolytica
MPPITQQATVTAWLPQVDASQITGTISSLESFTNRFYTTTSGAQASDWIASEWQALSASLPNASVKQVSHSGYNQKSVVMTITGSEAPDEWIVIGGHLDSTIGSHTNEQSVAPGADDDASGIAAVTEVIRVLSENNFQPKRSIAFMAYAAEEVGLRGSQDLANQYKSEGKNVVSALQLDMTNYKGSAQDVVFITDYTDSNFTQYLTQLMDEYLPSLTYGFDTCGYACSDHASWHNAGYPAAMPFESKFNDYNPRIHTTQDTLANSDPTGSHAKKFTQLGLAYAIEMGSATGDTPTPGNQ

SEQ ID NO: 11 - protein, 317 aa, Serratia marcescens
MEQLRGLYPPLAAYDSGWLDTGDGHRIYWELSGNPNGKPAVFIHGGPGGGISPHHRQLFDPERYKVLLFDQRGCGRSRPHASLDNNTTWHLVADIERLREMAGVEQWLVFGGSWGSTLALAYAQTHPERVSEMVLRGIFTLRKQRLHWYYQDGASRFFPEKWERVLSILSDDERKDVIAAYRQRLTSADPQVQLEAAKLWSVWEGETVTLLPSRESASFGEDDFALAFARIENHYFTHLGFLESDDQLLRNVPLIRHIPAVIVHGRYDMACQVQNAWDLAKAWPEAELHIVEGAGHSYDEPGILHQLMIATDRFAGK

SEQ ID NO: 12 - protein, 295 aa, Pyrococcus furiosus
MDTEKLMKAGEIAKKVREKAIKLARPGMLLLELAESIEKMIMELGGKPAFPVNLSINEIAAHYTPYKGDTTVLKEGDYLKIDVGVHIDGFIADTAVTVRVGMEEDELMEAAKEALNAAISVARAGVEIKELGKAIENEIRKRGFKPIVNLSGHKIERYKLHAGISIPNIYRPHDNYVLKEGDVFAIEPFATIGAGQVIEVPPTLIYMYVRDVPVRVAQARFLLAKIKREYGTLPFAYRWLQNDMPEGQLKLALKTLEKAGAIYGYPVLKEIRNGIVAQFEHTIIVEKDSVIVTTE

SEQ ID NO: 13 - protein, 793 aa, Lactobacillus helveticus
MKYNQYAYVETDFQQQVKELIDINFLPKNYQVWDFSSLLAKLVKNAIAEAKTDAAKNAKLAEFAVSDHQTLADFLKEKPTEIGTKQFYNVALQLLGYHVHYDYDFADPTGFMQRNALPFLQDISDNQKLISAFYRLLNTRAKNGQILLDVMAGKGYFTQFWGQNKFKFFNGKSIPVFDTNKVIREVVYVETDLDTDHDGKSDLIQVTVFRPEETNKGLKVPALYTASPYFGGIIANEKRNHNVDENLSDSTEWNDPQYVHSPIVKAEKPDGSSRPATEEAVHKSSYPLNEYMLARGFASVFAGAIGTRGSDGVRITGAPEETESAAAVIEWLHGDRVAYTDRTRTVQTTADWCNGNIGMTGRSYLGTLQIAIATTGVKGLKTVVSEAAISSWYDYYREHGLVIAPEACQGEDLDLLAETCQSNLWDAGSYLKIKPEYDKMQKQLREKEDRNTGQYSDFWEARNYRHHADGIKCSWISVHGLNDWNVKPKNVYKIWQLVKKMPMKHHLFLHQGPHYNMNNLVSIDFTDLMNLWFVHELLGIENNAYNQWPTVMIQDNLQADKWHEEPDWSNDLGQEKIYYPTDEGELFQDGNGKAQKSFTDVGGIEFKKAGISESDWQYKFICGDEKWAKPSLRFETDEFTHPTTIVGRPEVKVRVSASLPKGEISVALVELGERQRLTATPKFLMHGGQELGYRFGTDTLQEFVPDKKTKAKLITKAHMNLQNFKDMKKPEAIDADKFYDLDFLLQPTYYTIPSGSKLALIIYSTDQGMTKRPLEDETYTIDLANTEIKFYEK

SEQ ID NO: 14 - protein, 569 aa, Streptomyces griseus
MGRTTLVEHDVPVEMRDGTVLRADVWRPADGPPSPAVLFRTPYGKSPLGLATLTPAQCVDRGYAAVVQDTRGRFGSEGEWAPLDWSREGPDGYDTVEWTAGQPWCDGNVAMAGTSYQAIVQWLAAMEKPPHLRAIAPTMSTSAPFDAEQLGGSLRLDHLTSWLGLTALEWVQRRAAAGDPVDGAVVAEVVQLLTTPDVPLRRWPLSTILDFEGFPGRLRDIFAGKVATVADYRLGEVGVPTFSVGGWYDVFSHGTIELHRAMRAQDPVAGRHELVVGPWVHSGQLPQVQGEVNTGPYGSAQGARLADLHLDFFDRHLRPSAADAGTAAGGDVRYFLFGDDTWHRAESWPPPEAVDSPWYLAGPEGGEEDGRLLPEPPQEGFGQDAFTYDPENPVPSHGGRVLQLGRLAAGPLNQAHLEARPDVLLYTSEPLTEPLDVVGRQRLRLRFGSDAPATAVVAKLTDVHPDGRSLLVAEASLRLDTGNGVPPDEPYDADLLLGDTAWRFAPGHRLRVHVTSSNFPHLDRHPNVPGPVGEAEHCRPARQTVWYGGPYESVLRLRVLPAPSTGGIA

SEQ ID NO: 15 - protein, 15 aa, Artificial Sequence
  arbitrary example sequence string
  misc_feature (1)..(15): Xaa can be any naturally occurring amino acid
XXXXXXXXXXXXXXX

SEQ ID NO: 16 - protein, 12 aa, Artificial Sequence
  Test peptide
NNGGNNGGRGNK

SEQ ID NO: 17 - protein, 8 aa, Artificial Sequence
  peptide array
  misc_feature (1)..(2): Xaa can be any naturally occurring amino acid
XXGGNNGG

SEQ ID NO: 18 - protein, 7 aa, Artificial Sequence
  Peptide array
  misc_feature (1)..(1): Xaa can be any naturally occurring amino acid
XGGNNGG

SEQ ID NO: 19 - protein, 18 aa, Artificial Sequence
  Figure 14 peptide
HEVVENLLNYCFQTFLDK

SEQ ID NO: 20 - protein, 7 aa, Artificial Sequence
  Figure 14 peptide
FQTFLDK

SEQ ID NO: 21 - protein, 10 aa, Artificial Sequence
  Figure 14 peptide
HEVVENLLNY

SEQ ID NO: 22 - protein, 14 aa, Artificial Sequence
  Figure 15 peptide
GAGSSEPVTGLDAK

SEQ ID NO: 23 - protein, 13 aa, Artificial Sequence
  Figure 15 peptide
AGSSEPVTGLDAK

(19) United States
(12) Patent Application Publication
(10) Pub. No.: US 2022/0214354 A1
(43) Pub. Date: Jul. 7, 2022

(54) MEANS AND METHODS FOR SINGLE MOLECULE PEPTIDE SEQUENCING

(71) Applicants: VIB VZW, Gent (BE); UNIVERSITEIT GENT, Gent (BE)

(72) Inventors: Nico Callewaert, Nevele (BE); Simon Devos, Sint-Michiels (BE); Sven Eyckerman, Nazareth (BE); Joel Vandekerckhove, Loppem (BE)

(21) Appl. No.: 17/600,437

(22) PCT Filed: Apr. 1, 2020
(86) PCT No.: PCT/EP2020/059250

§ 371 Date: Sep. 30, 2021

(30) Foreign Application Priority Data
Apr. 3, 2019 (GB) 1904697.8

(51) Int. Cl. G01N 33/68

(52) U.S. Cl. CPC G01N 33/6824

(57) ABSTRACT

The present application relates to the field of protein sequencing, more particularly to protein profiling using massively parallel sequencing with single-molecule sensitivity. Methods, assays and reagents are provided for sequencing individual protein or polypeptide molecules. Also provided are methods and assays for the parallel sequencing of proteins or polypeptides. To this end, particular labeled probes are used that are reactive with the N-terminal amino acid of the polypeptide molecules and can be detected while still associated with the polypeptide(s).

### CROSS-REFERENCE TO RELATED APPLICATIONS

This application is a national phase entry under 35 U.S.C. § 371 of International Patent Application PCT/EP2020/059250, filed Apr. 1, 2020, designating the United States of America and published in English as International Patent Publication WO 2020/201350 on Oct. 8, 2020, which claims the benefit under Article 8 of the Patent Cooperation Treaty to United Kingdom Patent Application Serial No. 1904697.8, filed Apr. 3, 2019, the entireties of which are hereby incorporated by reference.

### FIELD OF THE INVENTION

The present application relates to the field of protein sequencing, more particularly to protein profiling using massively parallel sequencing with single-molecule sensitivity. Methods, assays and reagents are provided for sequencing individual protein or polypeptide molecules. Also provided are methods and assays for the parallel sequencing of proteins or polypeptides. To this end, particular labeled probes are used that are reactive with the N-terminal amino acid of the polypeptide molecules and can be detected while still associated with the polypeptide(s).

### BACKGROUND

Standard protein sequencing methods rely on the sequential detection of individually cleaved N-terminal amino acids using Edman degradation chemistry. The released N-terminal amino acid derivatives can then be identified using different chromatographic techniques. To overcome significant limitations of conventional Edman sequencing, several concepts for next-generation protein sequencing propose to determine the nature of the N-terminal amino acid while still attached to the protein. It has been suggested to use a set of labeled N-terminal amino acid binding proteins (NAABs e.g. antibodies or binders derived from tRNA synthetases or aminopeptidases) specifically binding (PITC-derivatized) N-terminal amino acids (WO2010065531; WO20140273004). As these proposed methods are compatible with the Edman degradation chemistry, but use a different read-out for identifying the N-terminal amino acid, Edman degradation can still be used to cleave off the N-terminal amino acid after it has been detected and identified. The identification-cleavage cycle can thus also be repeated. A drawback of said methods is that they rely on an arsenal of NAABs to derive amino acid identity information. NAABs for all different amino acids should be present together or added sequentially, adding complexity to this system. Moreover, the ability to develop NAABs with sufficient affinity to be used in single molecule sensing remains undemonstrated. Consequently, it would be advantageous to develop a more simple and elegant protein sequencing technology based on different physiochemical principles than mere binding affinity of reagents.

### SUMMARY

Here, an alternative single molecule peptide sequencing method is described. It is an object of the invention to provide methods that allow the simultaneous parallel sequencing of large numbers of polypeptides present in a given sample by sequencing amino acids still attached to the polypeptide, so as to allow attribution of a specific amino acid residue to a particular polypeptide present in the sample. This is made possible by the surprising finding that the kinetics of association and subsequent dissociation of an N-terminal amino acid binder, more particular of a chemical probe (e.g. a crown ether or a derivative thereof) depends on the N-terminal amino acid it is associated with. The different association (or binding) and dissociation (or detaching) specifics of a crown ether or derivative thereof (e.g. residence time of the crown ether on a protein's N-terminus) can thus be used to stepwise identify or categorize N-terminal amino acids of surface immobilized polypeptides. This allows the use of only one probe to identify different amino acids, particularly up to all different amino acids, which is both economical and beneficial in terms of user friendliness.

In order to monitor the binding kinetics of N-terminus binding probes such as crown ethers with consecutive amino acids from a polypeptide chain, the N-terminal amino acid has to be removed in a cyclic manner. After monitoring the binding kinetics on an N-terminal amino acid, the amino acid can be removed either enzymatically by an aminopeptidase or chemically by Edman degradation, after which the binding kinetics can be monitored on the next (now N-terminal) amino acid.

In a combination with Edman degradation, the crown ether binding kinetics is monitored at low pH (protonated N-terminal amine). When changing to high pH, the N-terminal amine gets deprotonated and the crown ether interaction is stopped. Then PITC is added which couples to the N-terminus. Finally, by changing back to low pH the N-terminal amino acid is cleaved (Edman degradation), and the crown ether can again bind to the free, protonated N-terminus (FIG. 8). Said combination might benefit from the use of (micro)fluidic systems to automatically cycle between high and low pH, for PITC coupling and PTH-amino acid cleavage respectively. As such the process proceeds in a more controlled manner, and because it entails a chemical method, higher temperatures and higher concentrations of salts or organic solvents can be used for denaturing the polypeptide's secondary structure.

Combining the crown ether binding kinetics method described herein with an aminopeptidase imposes stricter conditions in terms of temperature, pH and denaturing salts/organic solvents. However, the use of thermophilic aminopeptidases allows denaturing conditions to some extent. The advantage of using enzymatic removal of N-terminal amino acids is that the polypeptide chain can be processed and sequenced in a “one-pot” setup. At pH 7, the polypeptide N-termini will be largely protonated, and thus the crown ether binding kinetics can be monitored. When the aminopeptidase cleaves the N-terminal amino acid, the crown ether binding kinetics can be monitored on the next amino acid (FIG. 9).

In one aspect, the application provides methods for sequencing a polypeptide immobilized on a surface via its C-terminus, said method comprising:
a) contacting said surface immobilized polypeptide with a labeled probe, wherein the probe associates with the N-terminal amino acid of said polypeptide;
b) measuring the association and/or dissociation kinetics of said probe on said N-terminal amino acid, wherein comparing said association and/or dissociation kinetics to a set of association and/or dissociation reference values characteristic for said probe and a set of N-terminal amino acids allows identification of the N-terminal amino acid associating with said probe;
c) cleaving the N-terminal amino acid of the polypeptide; and
d) repeating steps a) to c) to determine the sequence of at least a portion of the polypeptide.

In one embodiment, said polypeptide is immobilized on a surface via a peptide moiety C-terminal to the first peptide bond of said polypeptide. The methods of the application are provided wherein said association and/or dissociation kinetics are measured optically, electrically or plasmonically. In a particular embodiment, said labeled probe is a fluorescently labeled probe and accordingly said association and/or dissociation kinetics are measured optically, more particularly fluorescently.

In other embodiments, said probe is a crown ether or derivative thereof, particularly an 18-crown-6 ether or derivative thereof. In yet other embodiments, the N-terminal amino acid of the polypeptide is chemically cleaved by isothiocyanate or isothiocyanate analogues or enzymatically cleaved by an aminopeptidase.

In another aspect, uses of crown ethers or derivatives thereof are provided to obtain sequence information of a polypeptide, more particularly to identify or categorize the N-terminal amino acid of said polypeptide. In one embodiment, said polypeptide is immobilized on a surface via its C-terminus. In another embodiment, the residence time of said crown ether or derivative thereof on the N-terminal amino acid of said polypeptide identifies or categorizes said N-terminal amino acid. In particular embodiments, said crown ether is an 18-crown-6 ether or derivative thereof, even more particularly a labeled 18-crown-6 ether or derivative thereof. Said label may be partly or wholly integrated in the crown structure.

In yet another aspect, a kit is provided comprising an 18-crown-6 ether or derivative thereof and an Edman degradation agent, more particularly ITC or ITC analogues. In another aspect, a kit is provided comprising an 18-crown-6 ether or derivative thereof and an aminopeptidase. Said kit are particularly suitable for the purpose of protein sequencing, more particularly for single molecule peptide sequencing.

### BRIEF DESCRIPTION OF THE FIGURES

FIG. 1 shows the immobilization of the Cy5 labeled test peptide (pepCy5) on a glass surface. Left, Control; Middle, pepCy5 at a concentration of 1 nM; Right: zoom image showing the successful spatial distribution.

FIG. 2 shows the trypsin digestion (1 nM) of surface immobilized peptides (1 nM pepCy5). A, The trypsin reaction on immobilized pepCy5 in absence of passivator. B, Trypsin treatment on immobilized pepCy5 in the presence of the passivator dbco-peg8-amide (1 μM). Signal was detected at 639 nm (upper panels) and background was assessed in the lower λ_{Em} channel of 561 nm (Cy3 channel) (lower panels).

FIG. 3 illustrates the structure of the triaza-18-crown-6 ether.

FIGS. 4A and 4B show observed NMR shifts of both Ala-methyl groups (A) and of the N-terminal Ala-methyl group alone at high resolution (B) of the peptide Ala-Ala-Phe with increasing crown ether concentration, indicative for a high crown ether exchange rate.

FIG. 5 illustrates the structures of 4′-aminobenzo-18-crown-6 ether (A) and 4′-aminodibenzo-18-crown-6 (B) ether.

FIGS. 6A-6D show the successful conjugation of 4′-aminobenzo-18-crown-6 ether (A2B) and Cy5.

FIG. 7 is schematic representation of the Edman degradation mechanism. Edman degradation entails the coupling of phenyl isothiocyanate (PITC) onto the free N-terminus of a protein/peptide (alkaline conditions), followed by the release of the N-terminal amino acid as a phenylthiohydantoin (PTH) derivative (acidic conditions). The released PTH-amino acid is then identified with chromatography. The procedure is then continually repeated leading to protein/peptide sequence information (source: https://en.wikipedia.org/wiki/Edman_degradation).

FIG. 8 is a schematic representation of the crown ether mediated polypeptide sequencing comprising Edman degradation steps.

FIG. 9 is a schematic representation of the crown ether mediated polypeptide sequencing comprising enzymatic removal of N-terminal amino acids.

FIG. 10A shows the interaction of Cy5.5-labeled dibenzo-18-crown-6 on a peptide array comprising 400 different peptides with all amino acid combinations at the first and second N-terminal position ([AA1][AA2]GGNNGG). FIG. 10B quantitatively illustrates the interaction between a selection of peptides and the labelled crown ether.

FIG. 11 illustrates the isothiocyanate-based method for C-terminal immobilization of peptides generated from protein digestion with LysC endoproteinase.

FIG. 12 illustrates a method for cleaving proteins after aminoethylated cysteines to generate longer peptides.

FIG. 13 shows the shifts in chromatogram upon conjugating the peptide GAGSSEPVTGLDAK with propargyl-isothiocyanate at both termini (middle) and then upon cleaving the conjugated N-terminal amino acid (bottom).

FIG. 14 shows the conversion of cysteine in HEVVENLLNYCFQTFLDK to S-aminoethyl-cysteine (middle) and the subsequent cleavage of the peptide by LysC endoproteinase (bottom).

FIG. 15 shows the ITC-based C-terminal conjugation of the peptide GAGSSEPVTGLDAK with azidophenyl isothiocyanate (N3-PITC) (middle) and the simultaneous coupling of N3-PITC and DBCO-PEG4-biotin resulting in a biotin C-terminal conjugated peptide (bottom).

FIG. 16 demonstrates the immobilization and single molecule detection of propargyl-ITC conjugated peptide. The peptide GAGSSEPVTGLDAK was conjugated with propargyl at the C-terminal lysine side chain using the ITC-based conjugation strategy, after which the free N-terminus was labeled with sulfo-Cy5. The peptide was subsequently immobilized on an azide surface using copper-catalyzed alkyn-azide cycloaddition (CuAAC). No single molecule spots were detected when either no peptide or unconjugated peptide were used (no immobilization; first and second picture from left), or when the peptide was conjugated but not treated with TFA (no sulfo-Cy5 labeling due to blocked N-terminus; third picture from left). Only when the peptide was conjugated and treated with TFA, single molecule spots were detected (right picture). The top and bottom row represents two separate technical replicate analysis.

FIG. 17 demonstrates single molecule detection of propargyl-conjugated peptides. The top pictures show single molecule peptide signals of the propargyl-conjugated peptide GAGSSEPVTGLDAK immobilized through CuAAC (3 technical replicates). Below each picture is a bleaching curve of one selected single. The single, discrete drop in intensity verifies that indeed a single molecule (peptide) is detected.

### DETAILED DESCRIPTION

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

### Definitions

The terms or definitions provided herein solely aid in the understanding of the invention. Unless specifically defined herein, all terms used herein have the same meaning as they would to one skilled in the art of the present invention. Practitioners are particularly directed to Michael R. Green and Joseph Sambrook, Molecular Cloning: A Laboratory Manual, 4^{th} ed., Cold Spring Harbor Laboratory Press, Plainsview, New York (2012); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

The term “probe” as used herein refers to a compound or molecule that selectively associates with (part of) another compound or molecule, not necessarily of the same nature. In the context of the present invention, a probe will associate with polypeptide molecules, typically with the N-terminal amino acid of the polypeptide molecules (and, according to particular embodiments, not with other amino acids than the N-terminal one, i.e. it will associate selectively with the N-terminal amino acid). According to particular embodiments, only one probe is used to allow detection of different amino acids. The phrase “associates with” or the term “association” as used throughout the application, refers to a more or less stable interaction between the molecule (polypeptide/amino acid) to be detected and the probe, which requires a certain proximity between the two. More or less stable means that the interaction is sufficiently stable to allow detection, the required stability of the interaction will typically depend on the manner of detection. Typically, such association will be achieved by the probe binding to the molecule (polypeptide/amino acid) to be detected, either by covalent or non-covalent binding, and “binds to” can in most cases be used as a synonym for “associates with”. However, association of the probe with the polypeptide can also be achieved without an actual chemical bond being formed, e.g. by electrostatic interaction. Other ways of associating may be envisaged as well, as long as the resulting interaction is stable enough to allow detection of the probe associated with the molecule (polypeptide/amino acid) to be detected. A non-limiting example of said probe is a crown ether, more particularly an 18-crown-6 ether (see specification).

A “labeled probe” as used herein is a probe carrying a detectable label. Although this typically implies the use of existing probes fused to a detectable label, the use of probes where the same moiety ensures specific recognition and functions as a label is also explicitly envisaged. A “detectable label” as used herein means any label that can be detected by using e.g. enzymatic, chemical, fluorescent, luminescent, electromagnetic or radioactive detection methods. According to particular embodiments, the label is a fluorescent label or a UV-detectable label, most particular a fluorescent label. Examples of labels will be provided in the specification. According to particular embodiments, a labeled probe may carry more than one label, for instance two, three or more labels. These labels may be identical (e.g. to strengthen the signal), may be different but of the same nature (e.g. two different fluorescent labels), may be of a different nature (e.g. a fluorescent and an electromagnetic label) or combinations thereof (e.g. two identical fluorescent labels and a third different fluorescent label). According to very specific embodiments, the probe may carry two labels that can be used for FRET detection (energy transfer between two chromophores) when associated with the N-terminal amino acid, whereby the nature and/or intensity of the FRET signal or signal change allows identification of the amino acid. This may also apply to BRET, photobleaching FRET or bimolecular fluorescence complementation (BiFC) detection.

The term “polypeptide” refers to polymers formed from the linking, in a defined order, of amino acids, in particular to two or more amino acids linked together by a peptide bond. As used herein, the term does not imply a length restriction: peptides, oligopeptides as well as proteins are encompassed within the definition. However, typically the polypeptides that are sequenced using the methods provided herein are at least 5 amino acids, at least 10 amino acids, at least 20 amino acids or at least 30 amino acids in length. Both synthetic and naturally occurring polypeptides can be used with the methods of the invention. “Sequencing at least a portion of the polypeptide” as used herein means that at least one (usually N-terminal) amino acid can be identified. However, typically more than one amino acid will be identified, e.g. at least two amino acids, at least 3 amino acids, at least 4 amino acids, at least 5 amino acids, and so forth. Ideally, sequencing will continue till a (N-terminal) portion of the polypeptide sequence has been characterized that is unique to the original protein/polypeptide, allowing identification of the original protein/polypeptide by comparing the characterized sequence with known sequences in a protein database. Depending on the nature of the sequence, a unique sequence will be obtained when 6 amino acids have been sequenced, 7 amino acids, 8 amino acids, 9 amino acids, 10 amino acids. If it is assumed that protein sequences are completely random, a sequence of 10 specific amino acids occurs only once in 20¹⁰ (10,240,000,000,000) possibilities. While protein sequences are not completely random, it is clear that in many, if not most cases a protein can be identified by characterizing a sequence of 10 or less amino acids. Optionally however, a portion of the polypeptide includes at least 10, at least 20, at least 30 or at least 50 amino acids. “At least a portion” also envisages the sequencing of the whole polypeptide. While sequencing typically will identify consecutive amino acids of the complete amino acid sequence, it is also envisaged that the identified sequence contains gaps, e.g. where a particular amino acid could not be resolved. According to particular embodiments, the length of the gap in the sequence is known. This can e.g. be derived from the number of cleavage (e.g. Edman) cycles performed, and as long as enough amino acids are identified, a sequence containing gaps can still be linked to the protein from which it is derived. Thus, “sequencing” as used herein includes partial sequencing.

The production of proteins or polypeptides is important to understand protein structures. Proteins are created by ribosomes that “read” RNA that is encoded by codons in the gene and assemble the requisite amino acid combination from the genetic instruction, in a process known as translation. The newly created protein strand then undergoes posttranslational modification, in which additional atoms or molecules are added, for example copper, zinc, or iron. Once this post-translational modification process has been completed, the protein begins to fold (sometimes spontaneously and sometimes with enzymatic assistance), curling up on itself so that hydrophobic elements of the protein are buried deep inside the structure and hydrophilic elements end up on the outside. The final shape or structure of a protein determines how it interacts with its environment. As such, proteins have a primary structure (i.e. the sequence of amino acids held together by covalent peptide bonds), secondary structure (i.e. regular repeating patterns such as alpha-helices and beta-pleated sheets), tertiary structure (i.e. covalent interactions between amino acid side-chains such as disulfide bridges between cysteine groups) and quaternary structure (i.e. protein sub-units that interact with each other). However, for the methods disclosed in the application, the protein and its N-terminal amino acid should be accessible for the labeled probe (e.g. crown ether or derivative thereof) of the application and preferably the protein is immobilized in a linear configuration. Therefore, in various embodiments, the protein to be sequenced is to be denatured. Denaturation is a process in which proteins lose the quaternary structure, tertiary structure and secondary structure, which is present in their native state, but the peptide bonds of the primary structure between the amino acids are left intact. Protein denaturation can be achieved by applying external stresses or compounds such as a strong acid or base, a concentrated inorganic salt, an organic solvent (e.g., alcohol or chloroform), radiation or heat.

The “N-terminal amino acid” or “aminoterminal amino acid” as used herein refers to the most N-terminal amino acid, amino acid residue or derivative thereof that is present in the polypeptide chain. It has a free amine group and is only linked to one other amino acid in the polypeptide, by a peptide bond. Derivatives of N-terminal amino acids are included within the definition. A derivative of an N-terminal amino acid is an N-terminal amino acid residue that has been chemically modified, for example by an Edman reagent or other chemical in vitro, or inside a cell via a natural post-translational modification mechanism, such as phosphorylation. Derivatives are used in the methods, as it e.g. may be necessary to block side chains of specific amino acid residues to prevent cross-reaction. Note for instance that all lysine side chains will be automatically blocked in the course of the first Edman cycle (as their PTC derivatives in case PITC is used). Another example of a derivative of an amino acid is a derivative that increases affinity of the probe-amino acid association. For instance, it may be that the probe has a higher affinity for the phenylthiocarbamyl derivative of the amino acid that is obtained when the polypeptide reacts with PITC in the course of an exemplary Edman reaction.

“To affix” as used throughout the application is the same as “to fix” and means establishing a connection between a polypeptide and a substrate such that at least a portion of the polypeptide and the substrate are held in physical proximity. Both indirect and direct connections, as well as reversible and irreversible connections are envisaged with this term.

The phrase “cleaving the N-terminal amino acid of the polypeptide” as used herein refers to a chemical reaction wherein the N-terminal amino acid is removed from the polypeptide while the remainder of the polypeptide molecule remains affixed to the substrate. A typical example where such cleavage occurs is during the Edman degradation reaction.

“Edman degradation” as used herein refers to the well-known chemical technique that allows sequential N-terminal degradation of a polypeptide and can therefore be used in N-terminal sequencing of proteins. It was first described by Pehr Edman in 1950, and in 1967 the degradation reaction was fully automated. Briefly, Edman degradation typically comprises two steps, a coupling step and a cleaving step (FIG. 7). These steps may be iteratively repeated, each time removing the exposed N-terminal amino acid residue of a polypeptide. In general, the coupling step of Edman degradation involves contacting the polypeptide with phenylisothiocyanate (PITC) or a suitable analogue thereof at an elevated pH (basic environment), thereby forming an N-terminal phenylthiocarbamyl derivative (in the case of PITC) or the like. Lowering the pH, e.g. by addition of an acid such as trifluoroacetic acid (TFA), results in the cleaving of the N-terminal amino acid derivative from the polypeptide to form a free anilinothiozolinone (ATZ) derivative or the stable phenylthiohydantoin (PTH) derivative (in both cases: if PITC was used initially) (FIG. 7). It is to be understood that the reagents suitable for Edman degradation are not limited to the Edman reagent PITC as many other compounds are known that can be used in the Edman degradation reaction. Suitable examples are included in the specification.

“Derivative” is derived from “derivatization” which refers to a technique used in chemistry or a mechanism of biochemistry which transforms a chemical compound into a product (the reaction's derivate) of similar chemical structure, called a derivative. Generally, a specific functional group of the compound participates in the derivatization reaction and transforms the product to a derivate of deviating, reactivity, solubility, boiling point, melting point, aggregate state, chemical composition, interaction or optical, electrical or plasmonic characteristics.

As used herein, the term “sample” refers to any material that contains one or more polypeptides (the polypeptides can be identical or different). Sample is used in a broad sense herein and is intended to include a wide range of biological materials as well as compositions derived or extracted from such biological materials, as well as synthetic compositions. Biological samples may comprise, for instance, a body tissue or fluid such as but not limited to blood (including plasma and platelet fractions), spinal fluid, mucus, sputum, saliva, semen, stool or urine or any fraction thereof.

The sample may or may not undergo preparation prior to applying the methods described herein. It may for instance be pretreated to achieve higher purity, a higher concentration of polypeptides, a lower concentration of contaminants. Non-limiting examples of such treatments include chromatographic separations such as HPLC or nuclease treatments. Another example of pretreatment is the protection of reactive groups (e.g. blocking of cysteine side chains). This can be done using for instance carboxymethylation or through performic acid oxidation. The sample may also undergo digestion with a protease, cleaving the original polypeptides (typically after a specific residue). For instance, the sample may be treated with trypsin, which cleaves after lysine or arginine residues.

“Single-molecule” as used in single molecule manner or at a single molecule level or in single molecule experiment refers to the investigation of the properties of individual molecules. Single-molecule studies may be contrasted with measurements on an ensemble or bulk collection of molecules, where the individual behavior of molecules cannot be distinguished, and only average characteristics can be measured.

Single Polypeptide Sequencing Based on Binding Kinetics

In current application, Applicants describe a method for peptide sequencing using a multiple step approach in which the N-terminal amino acids are identified one by one. It was surprisingly found that labeled probes that associate with and dissociate from N-terminal amino acids of a polypeptide (i.e. associating with the N-terminal amino acid but not with other amino acids) can be used to identify the sequence of said polypeptide, using the association and/or dissociation kinetics of the probe with the N-terminal amino acids. In contrast to classical Edman-based sequencing where the released anilinothiohydantoin (ATZ) or phenylthiohydantion (PTH) amino acids are each time identified by for example chromatography, the herein described approach determines the nature of N-terminal amino acids while still attached to the protein. Preferably this is done while the protein is immobilized on a solid surface. Interestingly because the probes used herein are compatible with cleaving-inducible agents (including chemical agents such as Edman degradation agents or enzymatic agents such as aminopeptidases), the process can be repeated for the following amino acids. By sequentially identifying and then cleaving the N-terminal amino acids of a polypeptide, sequence information can be generated.

According to a first aspect of the application, methods, assays and reagents for sequencing proteins are provided herein. These methods are useful for sequencing single polypeptide molecules.

More precisely a method of sequencing a polypeptide is provided, said method comprises the steps of: a) contacting said polypeptide with a labeled probe; b) measuring the residence time of said probe on the N-terminal amino acid of said polypeptide or alternatively measuring the association and/or dissociation kinetics of said probe on said N-terminal amino acid; c) identifying or categorizing said N-terminal amino acid by said residence time or said association and/or dissociation kinetics; d) cleaving said N-terminal amino acid from said polypeptide and e) repeating the steps a) through d) one or more times. In one embodiment, said polypeptide is immobilized on a surface. It goes without saying that when said agent cleaves the N-terminal amino acid from said polypeptide that said polypeptide is immobilized on a surface by its C-terminus. In another embodiment, said method is a method of sequencing a surface-immobilized polypeptide at single molecule level.

More specifically, methods are provided for sequencing a polypeptide molecule, particularly a single polypeptide molecule, comprising the following steps:
a) contacting said polypeptide molecule with a labeled probe, wherein the probe associates with the N-terminal amino acid of the polypeptide;
b) measuring the association and/or dissociation kinetics of said probe with said N-terminal amino acid;
c) comparing said association and/or dissociation kinetics to a set of association and/or dissociation reference values characteristic for said probe and a set of N-terminal amino acids;
d) cleaving the N-terminal amino acid of the polypeptide; and
e) repeating steps a) to d) to determine the sequence of at least a portion of the polypeptide.

In one embodiment, step b) from the above method is replaced by measuring the residence time of said probe on said N-terminal amino acid and step c) is replaced by comparing set residence time to a set of reference residence time values characteristic for said probe and a set of N-terminal amino acids. Both methods allow the sequencing of a purified solution of a single protein, as well as single molecule identification of a protein.

According to a particular aspect of the invention, the association and/or dissociation specifics of the labeled probe with particular N-terminal amino acids depends on or is characteristic for the N-terminal amino acid the labeled probe is associated with. Hence, measuring association and/or dissociation kinetics of the labelled probe allows the identification of said N-terminal amino acid associated with said probe.

According to some embodiments, the labeled probe allows to differentiate some, but not all, amino acids; i.e. the labeled probe association and/or dissociation properties (e.g. residence time of the labeled probe on N-terminal amino acid) are identical for different amino acids. Probes that cannot distinguish every amino acid (e.g. only allow ambiguous identification for some amino acids) may still be used to determine partial sequence information for a polypeptide (e.g. with gaps, or with more than one amino acid possibility at a given position of the sequence).

According to particular embodiments, the probes are detectable with single molecule sensitivity, particularly when associated with a polypeptide molecule. According to very specific embodiments, detecting the identity of the N-terminal amino acid is not done using mass spectrometry methods.

By first fixing or immobilizing the polypeptide molecule to a substrate, the sequence of the immobilized polypeptide can be determined by iteratively detecting the detectable signal of the label particular to the labeled probe-amino acid association. If multiple polypeptides are fixed to specific, spatially resolved positions on the substrate, it is possible to determine the sequence of the multiple polypeptides by iteratively detecting the signal of the label at the same respective locations on the substrate.

Therefore, according to a second aspect, methods are provided for the simultaneous sequencing of a plurality of single polypeptide molecules, such as for the basis of massively parallel sequencing techniques. This allows sequencing, at least in part, of individual polypeptide molecules present in samples comprising a mixture or multitude of different proteins. The methods not only allow generating sequence information from complex samples (qualitative data), but also quantitative data (how often a particular protein is present in a sample) can be obtained.

Accordingly, to a further embodiment, methods are provided for sequencing a plurality of polypeptide molecules in a sample, comprising:
a) affixing the polypeptides in the sample to a plurality of spatially resolved attachment points on a substrate;
b) contacting the polypeptides with a labeled probe, wherein the probe associates with the N-terminal amino acid of each polypeptide;
c) measuring the association and/or dissociation kinetics of said probe with each polypeptide molecule, wherein comparing said association and/or dissociation kinetics to a set of association and/or dissociation reference values characteristic for said probe and a set of N-terminal amino acids allows identification of the N-terminal amino acid of each polypeptide present on said substrate;
d) cleaving the N-terminal amino acid of each polypeptide; and
e) repeating steps b) to d) to determine the sequence of at least a portion of each polypeptide.

In one embodiment, said polypeptide molecule is or polypeptide molecules are immobilized on a surface via its/their C-terminus or via a peptide moiety C-terminal to the first peptide bond of said polypeptide(s). In another embodiment, step c) from the above method is replaced by measuring the residence times of said probe on the N-terminal amino acid of each polypeptide, wherein comparing said residence times to a set of reference residence times values characteristic for said probe and a set of N-terminal amino acids allow identification of the N-terminal amino acids.

For probes in the context of the present invention, any reagent can be used that has enough specificity for N-terminal amino acids, can be suitably labeled and is compatible with chemical (e.g. Edman degradation reaction) or enzymatic (e.g. aminopeptidase action) N-terminal amino acid cleavage. According to particular embodiments of the invention, the probe selectively associates with the N-terminus (N-terminal amino acid) of the polypeptide, typically with its free amine group, but is not selective for a particular amino acid. As a consequence, the number of different probes required to identify all different amino acids is less than the number of different amino acids, most particularly only one probe is used to identify the different amino acids (or possibly most different amino acid in the case of partial identification). Identification in such cases occurs through differences in label properties associated with the different interactions with the labeled probe depending on the nature of the N-terminal amino acid. If more than one probe is used for identification, particularly only two, three or four probes will be used for practical reasons. Indeed, sequencing will then typically require that a first labeled probe associates with the N-terminal amino acid, detecting the first labeled probe associated with the affixed polypeptide(s), possibly (and/or partially) identifying the N-terminal amino acid, removing the first labeled probe without removing the N-terminal amino acid, contacting the amino-terminal amino acid with the second labeled probe, detecting this probe associated with the polypeptide and so on, until all probes have been sequentially contacted with the polypeptide(s). The cleavage of the N-terminal amino acid in such case cannot be done before the detection of the last different probe.

Apart from the probe itself having specificity for N-terminal amino acids, specificity for N-terminal amino acids may also be obtained or increased by blocking side chain groups (e.g. lysine side chain groups) or by derivatizing the N-terminal amino acid, so that the pool of suitable compounds is increased. Basically, in order to be compatible with Edman degradation, the probes need to associate with the N-terminal amino acid in a way that does not interfere with the coupling and cleaving steps during Edman degradation; or the probes can themselves function as reagent used in the coupling step of Edman degradation chemistry. The former can be achieved by associations that are reversible, such as a covalent or non-covalent bond that is broken prior to coupling the coupling reagent in the Edman degradation.

One of the core aspects of current application is the finding that association and/or dissociation kinetics between labeled probes and N-terminal amino acids of a polypeptide are informative for the identity of said N-terminal amino acids.

Association is the first phase in a biomolecular interaction experiment. The association rate constant Ka describes the rate of complex formation, e.g. the number of probe-peptide complexes formed per second in a one molar solution of probe and peptide. The units of Ka are M⁻¹ s⁻¹ and are typically between 1.103 and 1.107 in biological systems. Once binding has occurred, the probe and peptide remain bound together for a random amount of time. The dissociation rate constant Kd describes the stability of the complex, i.e. the fraction of complexes that decays per second. The unit of Kd is 1/s and is typically between 1.10⁻¹ and 1.10⁻⁶ in biological systems. A Kd of 1.10⁻²/s=0.01/s. This means that 1 percent of the complexes decay per second. The measurement of binding constants (Ka and/or Kd) allows thus the evaluation of the strengths of probe-peptide interactions and thus gives a quantitative means of comparing the binding properties of said probe or probes and its or their selectivit(y)(ies) for different N-terminal amino acids.

The time that for example the probe associates with the N-terminal amino acid from the immobilized peptide is referred to as the “residence time” or the “contact time” or the “on-time” of the labeled probe on the N-terminal amino acid. Hence, the “on-time” and “residence time” which will be used interchangeably herein refer to the time of a probe (e.g. labeled crown ether) acting on one peptide molecule until it spontaneously dissociates from the N-terminal amino acid. Said “on-time” of a probe such as an 18-crown-6 ether can in this case easily be determined by labelling said probe. As such the label acts as a proxy for the “on-time” of the probe and thus for the identity of the N-terminal amino acid to which the probe binds. In a particular embodiment of this application, said probe can be optically, fluorescently, electrically or plasmonically labelled (see later).

In alternative embodiments, the labeled probes used in the methods of said application can have several rounds of association on and dissociation from the N-terminal amino acids. Every residence time of said probes until a cleaving-inducing agent is added to cleave off the N-terminal amino acid will be informative to identify the N-terminal amino acid. Therefore, in order to predict the N-terminal amino acids more accurately in a single molecule set-up, it is recommended to have multiple residence time measurements for every probe-N-terminal amino acid association.

It is thus also envisaged that the step of measuring the residence time of the labeled probes in the methods of the application implies the measuring of multiple residence times of said probes before the N-terminal amino acid is cleaved off. Alternatively phrased, the methods of the application are provided wherein the residence time of said labeled probe is measured for every association event of said probe and said N-terminal amino acid.

In particular embodiments, the methods disclosed in current application are provided wherein the labeled probe on average has at least 3, at least 4, at least 5, at least 6, at least 7, at least 8, at least 9, at least 10, at least 20 or at least 50 association/dissociation cycles before a cleaving-inducing agent is added and the N-terminal amino acid is cleaved off.

According to particular embodiments, the N-terminal amino acid is cleaved from the polypeptide after the detection step took place. By cleaving, the subsequent amino acid in the sequence of the polypeptide becomes the new N-terminal amino acid (i.e. with an exposed N-terminus, and thus free to react with a labeled probe as described herein). In some embodiments, these steps may be iteratively repeated up until the last, i.e. C-terminal, amino acid of the polypeptide has been reached. According to alternative particular embodiments, the C-terminal amino acid remains affixed to the substrate, e.g. via a covalent attachment. According to particular embodiments, cleavage is done via Edman degradation, as this technique is well suited for sequential cleavage of N-terminal amino acids. According to other particular embodiments, cleavage is done enzymatically, as this technique allows cleavage of N-terminal amino acids in less harsh conditions (e.g. pH neutral conditions).

According to particular embodiments, the steps of adding the labeled probe, detecting the labeled probe associated with the polypeptide and cleaving the N-terminal amino acid are iteratively repeated so that sequence information on the polypeptide can be obtained. The steps may be repeated at least 2 times, more particularly at least 5, at least 10, at least 15, at least 20, at least 30, at least 40, at least 50 times, or even more in order to sequence a part of or the complete polypeptide. The identified amino acids may be contiguous, partly contiguous or discontiguous, and the amino acid sequence may be identified in full or partially. Preferentially, if the amino acids are not identified contiguously, the length of the gap in the amino acid sequence is determined, so that comparison with known sequences is facilitated. It is possible that an amino acid is partly identified, i.e. while the exact identity is not determined, the possible identities for a given amino acid in a sequence are narrowed down to a couple or a few amino acids.

Optionally, washing steps may be implemented in the methods described herein. Washing may occur before or after affixing the polypeptide on the substrate, and/or before or after adding the labeled probe, and/or before or after detecting the labeled probe associated with the polypeptide, and/or before or after the cleavage of the N-terminal amino acid, and/or before or after the optional coupling step if Edman degradation is used. Generally speaking, washing is done to remove impurities, contaminants or excess reagents, in order not to interfere with following steps in the procedure. In specific embodiments, washing may also be a step when changing the pH or buffer of the medium in contact with the substrate and/or polypeptide.

It is particularly envisaged that in some embodiments, the methods allow the sequencing of multiple polypeptide molecules in parallel. These polypeptides may be affixed to the same substrate, or to a plurality of substrates. In accordance with these embodiments, methods are provided for simultaneous sequencing of a plurality of affixed polypeptide molecules. Typically, this plurality of polypeptides will be present in (or derived from) one or more samples, such as those defined herein.

As with the methods using a single polypeptide, methods according to these embodiments may include comparing the obtained sequence from part or all of the polypeptides to known polypeptide sequences, e.g. as found in a reference protein sequence database. This can for example be done using BLAST or other suitable protein sequence comparing algorithms. According to particular embodiments, sequence fragments of the affixed polypeptide(s) are used for comparing, and so detecting the identity of the polypeptide(s). According to further particular embodiments, the fragments comprise 20 or fewer identified amino acids, 15 or less identified amino acids, 10 or less identified amino acids or 5 or less identified amino acids. Note that the actual query may contain more information than just the identified amino acids, such as information about sequence gaps or about partially identified amino acids. By way of an arbitrary example, such a sequence string could look like AAAXAYAAXYAAAAA, wherein A is a fully identified amino acid, X is a gap of one amino acid and Y is a partially or ambiguously identified amino acid, i.e. an amino acid that could not be unambiguously determined, but for which a number of possible identities have been ruled out.

According to alternative embodiments, the polypeptides are sequenced in full, and may or may not be compared to known sequences to determine the identity.

Where multiple polypeptide sequences are determined in parallel, this may also yield information about the occurrence of a particular polypeptide in a sample. Indeed, the sequence will be determined as often as the polypeptide is present on the substrate. According to particular embodiments, the nature and or number of the polypeptides present in the sample is used to learn more about the sample (i.e. the sample is analysed by quantitative and/or qualitative means). For example, specific proteins and/or the number of specific proteins can yield information on identity of (micro-)organisms, or about healthy or diseased state of a tissue.

Crown Ethers

In particular embodiments throughout current application, said probe is a chemical. In even more particular embodiments, said probe is a crown ether. A “crown ether” as used in the application refers to heterocyclic chemical compounds that consist of a ring containing several ether groups. The first number in a crown ether's name refers to the number of atoms in the cycle, and the second number refers to the number of those atoms that are oxygen. Crown ethers have the ability to form complexes with cations and small molecules such as hydrated proton ions and pronated amines, such as primary amines found in N-terminal amino acids. Although the most common crown ethers are oligomers of ethylene oxide, crown ethers are much broader than the oligomers of ethylene oxide, e.g. catechol-derived crown ethers. Included within the definition of crown ethers are derivatives that still contain the crown ether core (e.g. 18-crown-6 ethers).

Due to their unique binding properties, crown ethers have been suggested for many applications, for example as modulators of ion transport, as antibiologicals and as structural probes (Wu et al 2018 Chinese J Anal Chem 46:273-280). However, herein, Applicants describe for the first time the use of crown ethers, more particularly of 18-crown-6 ethers in single molecule protein sequencing. The invention is based on the finding that the kinetics of the association between a labeled 18-crown-6 ether and N-terminal amino acids and/or the kinetics of the dissociation of said labeled 18-crown-6 ether from N-terminal amino acids are informative for the identity of said N-terminal amino acid. Crown ethers are particularly suited for the methods of current application as crown ethers more particularly 18-crown-6 ethers have been shown to associate with the 20 common types of amino acids. Moreover, it has been demonstrated previously that the association and dissociation parameters of said 18-crown-6 ethers differ for different amino acids (see Wu et al 2018 Chinese J Anal Chem 46:273-280).

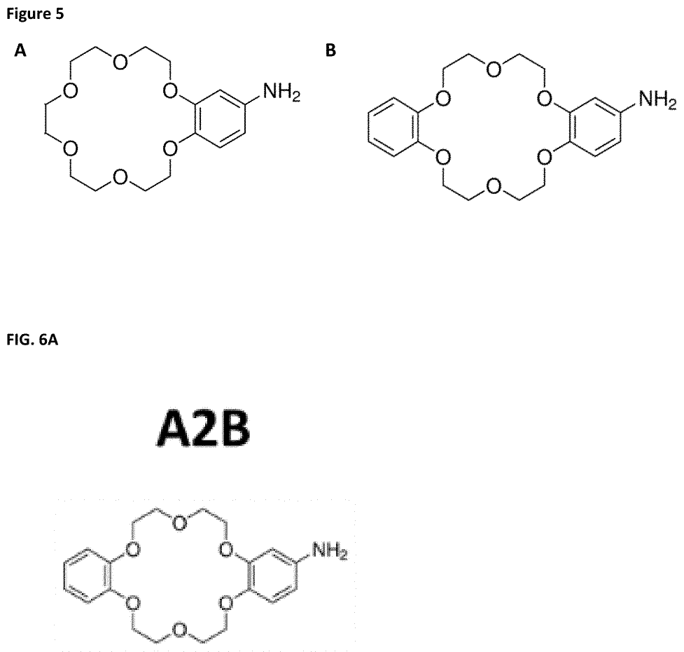

Particularly envisaged in current application are the 18-crown-6 ethers, as the cavity size is ideal for complexation with a primary amine, such as those encountered in N-terminal amino acids. According to particular embodiments, the crown ether is a 4′-aminobenzo-18-crown-6 ether or a 4′-aminodibenzo-18-crown-6 ether (FIG. 5).

According to alternative embodiments, the crown ether has one or more oxygen atoms that are substituted by nitrogen atoms. Particularly envisaged are mono-, di- and triaza crown ethers. A non-limiting example is 18-crown-O₃N₃ (triaza 18-crown-6), as this reagent was shown to have a high specificity towards primary amines (Lehn et al 1980 Tetrahedron Letters 21:1323-1326).

As described above, the application provides methods for sequencing polypeptides. In said methods, N-terminal amino acids from said polypeptides are sequentially cleaved after their identification based on association and/or dissociation kinetics with a labeled probe (e.g. a labeled 18-crown-6 ether). Cleaving the N-terminal amino acids can be achieved most standardly by chemicals (e.g. Edman degradation) or enzymatically by peptidases, more particularly aminopeptidases. Said cleavage-inducible agents (e.g. ITC, ITC analogues or aminopeptidases) can covalently or non-covalently bind to said N-terminal amino acids.

Crown Ether—Edman Degradation Compatibility

In various embodiments, the cleaving step in the methods of current application is performed by a cleavage-inducing agent. In a particular embodiment of this application, the cleavage-inducing agent referred to in the uses and methods of the application is a chemical agent, more particularly an Edman degradation agent selected from the list consisting of isothiocynanate (ITC), phenyl isothiocyanate (PITC), azido-PITC, coumarinyl-isothiocyanate (CITC), sulfophenyl isothiocyanate (SPITC), dimethylaminoazobenzene isothiocyanate (DABITC), naphtyl isothiocyanate (NITC), 3-pyridyl isothiocyanate (PYITC), 2-piperidinoethyl isothiocyanate (PEITC), 3-(4-morpholino) propyl isothiocyanate (MPITC), 3-(diethylamino) propyl isothiocyanate (DEPITC) and 2,3-Dihydroxymalemide-4′-phenylisothiocyanate (DHMPITC). In current application PITC, azido-PITC, DABITS, NITC, PYITC, PEITC, MPITC, DEPITC, DHMPITC, CITC and SPITC will be referred to as isothiocyanate analogues. Hence, the methods of current application are provided wherein the cleavage-inducing agent or Edman degradation agent is isothiocyanate (ITC) or an ITC analogue.

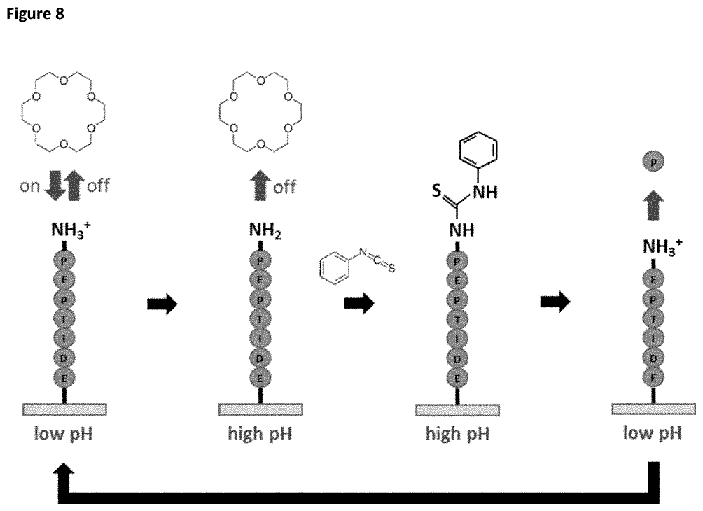

As described above, crown ethers bind to the protonated form of primary amines, an interaction that needs to be done at low pH. As coupling of ITC or ITC analogues occurs at basic pH and Edman degradation itself involves switching from basic to acidic pH and back again, the crown ether mediated identification step of the N-terminal amino acid is fully compatible with Edman degradation. Indeed, during the low pH step the crown ether will be allowed to associate with and dissociate from the N-terminal amino acid of the surface immobilized polypeptide for multiple times (FIG. 8). The kinetics of these steps (e.g. the residence times of the crown ether on the N-terminal amino acid) identify the N-terminal amino acid. Once enough information is gathered to identify or categorize said N-terminal amino acid, the pH is increased in a next step resulting in deprotonation of the N-terminus which terminates the association with the crown ether and allows Edman coupling (FIG. 8). In a next step, the pH is reduced again to cleave off the N-terminal amino acid in a classical Edman degradation. At the same time the new N-terminal amino acid will get protonated hence allowing crown ethers to associate again with the next amino acid to be identified (FIG. 8).

Accordingly, in alternative embodiments, the labeled probes herein described (e.g. the 18-crown-6 ether from the application) reversibly associate with the N-terminal amino acid of a polypeptide prior to a coupling step of an Edman degradation agent.

Crown Ether—Aminopeptidase Compatibility

In other embodiments, said cleavage-inducing agent is a peptidase, more particularly a catalytically active peptidase, even more particularly a catalytically active aminopeptidase. When an aminopeptidase is used in the methods of current application said polypeptide is surface-immobilized through its C-terminus.

“Aminopeptidase” as used herein refers to an enzyme that catalyzes the cleavage of amino acids from the amino terminus (N-terminus) of protein or peptide substrates. They are widely distributed throughout the animal and plant kingdoms and are found in many subcellular organelles, in cytosol, and as membrane components. Aminopeptidases are classified by 1) the number of amino acids cleaved from the amino terminus of substrates (e.g. aminodipeptidases remove intact amino terminal dipeptides, aminotripeptidases catalyze the hydrolysisis of amino terminal tripeptides), 2) the location of the aminopeptidase in the cell, 3) the susceptibility to inhibition by bestatin, 4) the metal ion content and/or residues that bind the metal to the enzyme, 5) the pH at which maximal activity is observed and 6) which is most relevant for this application by the relative efficiency with which residues are removed (Taylor 1993 FASEB J 7:290-298). Aminopeptidases can have a broad or a small substrate specificity. In this application the focus is on the use of broad substrate specificity aminopeptidases, however the use of multiple aminopeptidases with substrate specificities that overlap or are complementary are also envisaged in this application.

In contrast to the use of Edman degradation agents (see above), aminopeptidases enable to perform the polypeptide sequencing methods herein disclosed in neutral pH conditions. Those neutral pH conditions are mostly wanted to avoid degradation of equipment and to be compatible with for example fluidics systems.

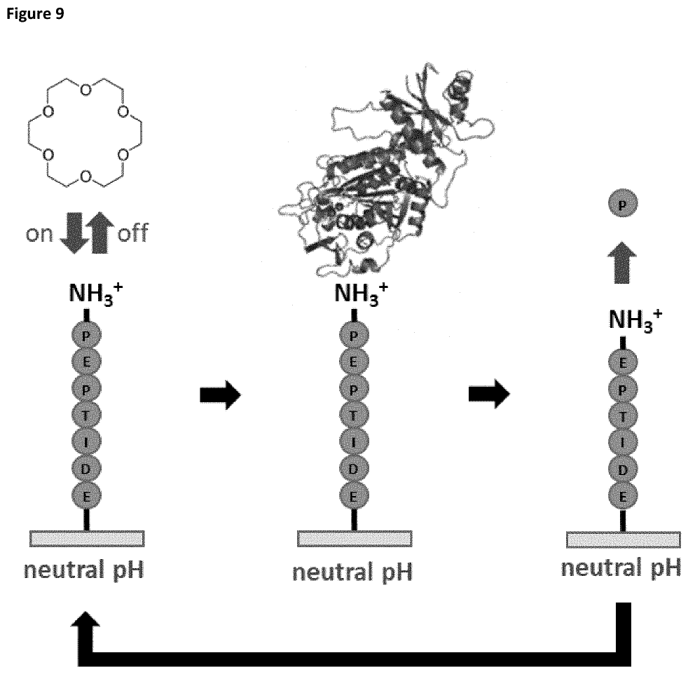

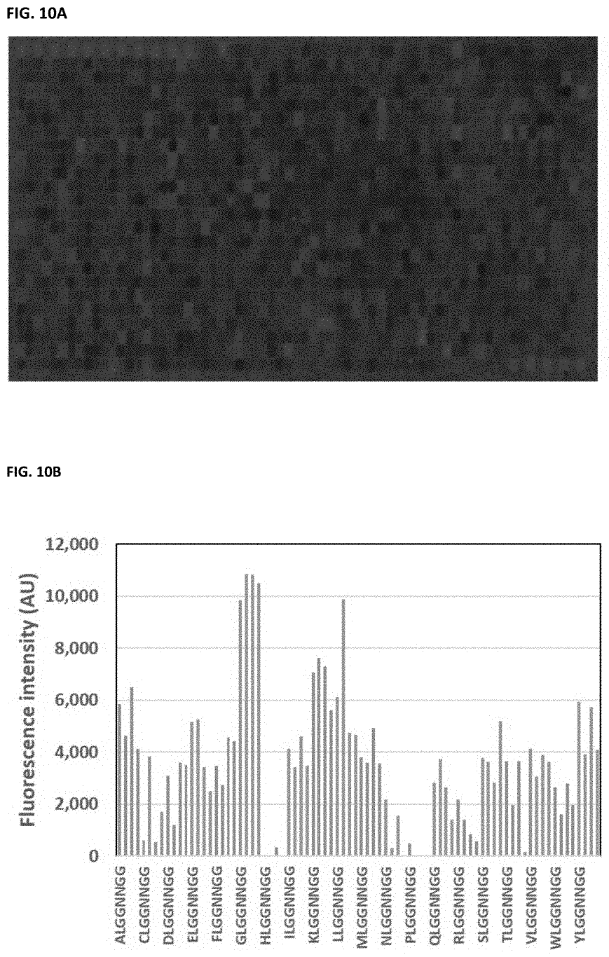

Moreover, the use of aminopeptidases in the methods of the application allows a “one-pot” reaction, i.e. sequencing of polypeptides by successive chemical reactions in just one reactor. Such strategy is especially desired for industrial applicability as it can save time and resources, e.g. it avoids washing away of solutions and buffers, intermediate adjustments of pH, separation processes and purification of the intermediate chemical compounds. At neutral pH allowing optimal activity of aminopeptidase, the N-terminal amino acids will be largely protonated, hence allowing association/dissociation of crown-ether with the N-terminal amino acids (FIG. 9).

Non-limiting examples of aminopeptidases suitable for the uses and methods described in current application are the aminopeptidase T from Thermus aquaticus (AMPT_THEAQ), aminopeptidase T from Thermus thermophilus (AMPT_TH ET8), PepC from Streptococcus thermophiles (PEPC_STRTR), Aminopeptidase S from Streptomyces griseus (APX_STRGG), Aminopeptidase from Streptomyces septatusTH-2 (Q75V72_9 ACTN), Aminopeptidase 2 from Bacillus stearothermophilus (AMP2_GEOSE) as well as the wild-type and engineered Trypanosoma cruzi cruzipain (or cruzain) and Thermus aquaticus aminopeptidase T as depicted in SEQ ID No. 1-8. Also suitable are Streptomyces griseus aminopeptidase (SGAP; UniProtKB-P80561) as depicted in SEQ ID No. 9, Aeromonas proteolytica aminopeptidase (APAP; UniProtKB-Q01693) as depicted in SEQ ID No. 10, Serratia marcescens aminopeptidase (SMAP; UniProtKB-032449) as depicted in SEQ ID No. 11, Pyrococcus furiosus aminopeptidase (PFAP; UniProtKB-P56218) as depicted in SEQ ID No. 12, Lactobacillus helveticus X-prolyl dipeptidyl aminopeptidase as depicted in SEQ ID No. 13 and Streptomyces griseus X-prolyl dipeptidyl aminopeptidase as depicted in SEQ ID No. 14.

Labelling of Probes and Detection Thereof

In order to detect and measure the “on-time” values or the residence time of the probe (e.g. crown ethers of derivatives thereof) on the N-terminal amino acid of an immobilized polypeptide, said probe needs to be detected. The nature of detection is not vital to the invention, as long as the probe “on-time” or the residence time of the probe can be detected. One way of detecting the probe of the application is by fusing it to a molecular label and subsequent detection of the molecular label. Accordingly, to a particular aspect of the invention, labeled probes are used, i.e. the probes include detectable labels. According to particular embodiments, the labels used are (or include, if more than one label is used) labels that can be detected as a single molecule.

Methods of labeling can be broken down into several categories depending on the label. The three main categories are potentiometry (the difference in electrode potentials is measured), coulometry (the current is measured over time), and voltammetry (the current is measured while the potential is actively altered). There are two basic categories of coulometric techniques. Potentiostatic coulometry involves holding the electric potential constant during the reaction using a potentiostat. The other, called coulometric titration or amperostatic coulometry, keeps the current (measured in amperes) constant using an amperostat. A non-limiting example of an electrical label is sulfophenyl isothiocyanate (SPITC). SPITC is a negatively charged variant of the phenyl isothiocyanate (PITC) probe that is used in MS de novo peptide sequencing for neutralizing N-terminal fragment ions (Samyn et al. 2004 J Am Soc Mass Spectrom 15:1838-1852). In certain embodiments of the application, the “on-time” of the probe is detected optically, electrically or plasmonically. In particular embodiments, electrically labeled can be potentiometrically, amperometrically or voltametrically labeled.

Optical detection requires optical labels and includes but is not limited to luminescent and fluorescent detection. The label can thus be a fluorophore. Commercially, there is an extensive catalog of optical labels available. According to particular embodiments, the probes (e.g. 18-crown-6 ethers) are fluorescently labeled. “Fluorescence” as used herein is the emission of electromagnetic radiation light by a substance that has absorbed radiation of a different wavelength. In most cases, absorption of light of a certain wavelength induces the emission of light with a larger wavelength (and lower energy). Note that fluorescence is not limited to visible wavelengths, as emission or absorption of UV, infrared and X-ray wavelengths is envisaged within the present application. Examples of fluorescent labels that can be used include, but are not limited to, fluorescein, Texas red dyes, Oregon green, rhodamine, coumarine, fluorescamine, dialdehydes such as o-phthalaldehydes (OPA) or naphthalene-2,3-dicarboxaldehydes (NDA), the Fmoc-reagent, the AccQ-Fluor reagent (Waters), 7-fluoro-4-nitrobenzo-2-oxa-1,3-diazole (NBD-F), dansyl chloride, Cy-3 and Cy-5 dyes, SiR dyes, GFP and GFP-variant labels (e.g. S65T GFP, EGFP, those described in EP851874), YFP and YFP-variant labels (e.g. Citrine, Venus, YPet), BFP and BFP-variant labels (e.g. EBFP, EBFP2, Azurite, mKalamal), CFP and CFP-variant labels (e.g. ECFP, Cerulean, CyPet), Cherry fluorescent protein, tdTomato, TMR, TAM RA, boron-dipyrromethene-based dyes (BODIPY), Alexa Fluor dyes, AMCA, Bimane, Cascade Blue dye, Cascade yellow dye, dapoxyl dye, Marina Blue dye, Pacific Blue dye, Pacific Orange dye, other commercially available dyes (e.g. from Molecular Probes), or a derivative or modification of any of these labels or dyes. Particularly envisaged are dyes with narrower emission bands (as these allow easier observation of a shift in emission spectrum), and/or dyes which are prone to emission shifts or intensity by presence of specific moieties or substituents (as these allow bigger PET in identifying the amino acid), and/or dyes whose spectrum is relatively insensitive to pH (as during Edman degradation pH changes repeatedly), and/or dyes which are amine reactive (as this can help in the probe aspect). An example of a dye that fits these different criteria is the BODIPY range of dyes. According to further particular embodiments, the fluorescent labels are integrated in the probe, e.g. FITC and other fluorescein isothiocyanate derivatives that can be used as Edman reagents, or crown ethers with a fluorophore integrated in the crown or ring structure, such as the fluorescent coumarin-labeled crown ether used by Nagy et al. (Nagy et al 2008 Tetrahedron 64:6191-6195). Integration of the label in the probe can also be used for non-fluorescent labels.

In one embodiment, the detecting step produces an image, e.g., a fluorescence image (e.g., acquired using Fluorescence Resonance Energy Transfer (FRET), Total Internal Reflection Fluorescence (TIRF), or Zero Mode Waveguide (ZMW)). In another embodiment, the compilation of the images makes a digital profile, e.g., a digital profile that identifies the immobilized polypeptide or its N-terminal amino acids. In particular embodiments, optically labelled is fluorescently labelled. In even more particular embodiments, fluorescent labels are measured or detected through TIRF microscopy.

Also, a plasmonic read out can be used to detect the “on-time” of the probe (e.g. crown ethers or derivatives thereof). In physics, a plasmon can be defined as a quantum for the collective oscillation of free electrons, usually at the interface between (noble) metals and dielectrics. The term plasmon refers to the plasma-like behavior of the free electrons in a metal under the influence of electromagnetic radiation. Surface plasmons are coherent delocalized electron oscillations that exist at the interface between any two materials where the real part of the dielectric function changes sign across the interface (e.g. a metal-dielectric interface, such as a metal sheet in air). The excitation of surface plasmons which can be done very efficiently with light in the visible range of the electromagnetic spectrum, is frequently used in an experimental technique known as surface plasmon resonance (SPR). In SPR, the maximum excitation of surface plasmons is detected by monitoring the reflected power from a prism coupler as a function of incident angle or wavelength. This technique can be used to observe nanometer changes in thickness, density fluctuations, or molecular absorption and is used for screening and quantifying protein binding events. Commercialized instruments are available that operate on these principles. Therefore, in particular embodiments, the “on-time” of the probe is determined by surface plasmon resonance.

However, notwithstanding the above, it must be clear that the nature of labelling and consequently detection is not vital to the invention, as long as the “on-time” or the residence time of the probe (e.g. crown ethers or derivatives thereof) can be detected.

In particular embodiments of current application, the association of the labeled probe (e.g. labeled 18-crown-6 ether) with the N-terminal amino acid will have an effect on detection of the labeled probe, depending on the nature of the particular amino acid with which the probe is associated. For example, binding of fluorescent crown ether compounds to the N-terminus of proteins may affect fluorescence intensity and fluorescence emission spectrum through photo induced electron transfer between the binding N-terminal amino acid and the fluorophore. The intensity and/or emission spectrum shift can be measured and correlated with the nature of the N-terminal amino acid the labeled probe is associated with. According to specific embodiments, the nature of the amino acid adjacent to the N-terminal amino acid has no influence on the signal properties of the label. According to alternative specific embodiments, the nature of the amino acid adjacent to the N-terminal amino acid does have an influence on the signal properties of the label. Ideally, this influence can be characterized so that the N-terminal amino acid can still be identified (optionally by taking into account the identification of said adjacent amino acid in turn).

Thus, in these embodiments, the N-terminal amino acid is identified by detecting the labeled probe in association with the N-terminal amino acid, as the signal of the label (or combination of labels) is influenced by the nature of the amino acid. It is a possibility that some labels will not allow discriminating between all amino acids (e.g. because the intensity and/or spectrum shift is not large enough, or is almost identical to the shift observed for other amino acids). However, distinguishing a number of particular amino acids is also useful, e.g. distinguishing 5 amino acids or more, 8 amino acids or more, 10 amino acids or more, 12 amino acids or more, 15 amino acids or more. Indeed, partial determination of a sequence may still allow identification of the original protein, as long as enough amino acids have been identified.

According to particular embodiments, the labeled probes associated with the N-terminal amino acid of a polypeptide affixed to a substrate are repeatedly detected at that location using a high resolution rastering laser/scanner. Detection can be across a predetermined grid, at a unique position or along a specific path on the substrate. According to further particular embodiments, the polypeptides are affixed randomly to the substrate; the detection of the labeled probes (and identification of the N-terminal amino acids) proceeds by repeatedly scanning the substrate to identify the coordinates of the labeled probes associated with the polypeptides fixed to the substrate. According to other particular embodiments, the detection is done using ultrasensitive detection systems that are able to repeatedly detect signals from exactly the same co-ordinates on a substrate, thereby allowing to assign the detected sequence information to a unique polypeptide or protein molecule affixed at those co-ordinates. According to yet further particular embodiments, the detection of the labeled probes is done using optical detection means. Such optical detection means or systems include, but are not limited to, a charge-coupled device (CCD), scanning microscopy means, confocal microscopy means, epi-illumination (e.g. using an epifluorescence microscope), light scattering means, dark field microscopy means, photoconversion means, total internal reflection fluorescence microscopy means, single or multiphoton excitation means, spectral wavelength discrimination means, fluorophore identification means, evanescent wave illumination means, and Stimulated Emission Depletion (STED) microscopy means. The techniques used are those for which the means or systems are adapted (e.g. confocal laser scanning microscopy, fluorescence lifetime imaging microscopy (FLIM), FRET, etc.). In general, detection may be done through laser-activated fluorescence using a microscope equipped with a camera (e.g. photodiodes, intensified CCD cameras).

Immobilisation and Labeling

“Immobilization on a surface” or “affixing on a surface” as used herein refers to the attachment of one or more polypeptides to an inert, insoluble material for example a glass surface resulting in loss of mobility of said polypeptides. For the methods disclosed in current application, immobilization allows the polypeptide(s) to be held in place throughout the sequencing of the polypeptide or identifying or categorizing the N-terminal amino acid of said polypeptide. The N-terminus should thus be freely accessibly, hence the polypeptide should be immobilized through its C-terminus. Moreover, proteins immobilized onto surfaces with high density allow the usage of small amount of sample solution. Many immobilization techniques have been developed in the past years, which are mainly based on the following three mechanisms: physical, covalent, and bioaffinity immobilization (Rusmini et al 2007 Biomacromolecules 8: 1775-1789; U.S. Pat. No. 6,475,809; WO2001040310; U.S. Pat. No. 7,358,096; U520100015635; WO1996030409; WO2013112745).

Chemical “click technologies” or “click chemistry” and biotin-(strept)avidin interactions (Kolb et al 2001 Angewandte Chemie-Int Ed 40:2004-2021) to immobilize peptides on a surface attracted a lot of interest due to its efficiency, versatility, and selectivity (Tron et al 2008 Med Res Rev 28:278-308). Also in this application polypeptides are immobilized on glass surfaces using the azide-dibenzocyclooctyl (DBCO) click reaction (see Example 1 and 2) according to protocols available in the art, e.g. Eeftens et al (2015 BMC Biophys 8:9).

However, click chemistry can only be used in the presence of click reaction partners. Surfaces can be coated by one or more of these click reaction partners, but linking them to biomolecules such as peptides is less common. For immobilization of synthetic peptides, the linking partners can easily be incorporated. Alternatively, recombinant fusion proteins have been proposed between the peptide to be immobilized and the linking partners (e.g. WO2003008453A1). For N-terminal conjugation it has been suggested to use isothiocyanate. For example, Shamsi et al (2011 Surface Science 605: 1763-1770) linked a peptide through its N-terminus to 4-azidophenyl isothiocyanate and subsequently attached the azido peptide to a silicon surface coated by alkyne-terminated monolayer. However, no solution is available to conjugate natural peptides obtained from a crude protein extract to a surface, particularly not for C-terminal conjugation onto a surface.

In this application a novel immobilization method is disclosed using the chemical isothiocyanate (ITC) (Examples 7-10). The method is based on the principle that each peptide comprises a free amino-group at the N-terminus and a free carboxyl-group at the C-terminus. From ITC and its analogues such as phenyl isothiocyanate (PITC), azido-PITC, coumarinyl-isothiocyanate (CITC), sulfophenyl isothiocyanate (SPITC) or fluorescein isothiocyanate (FITC) it is known that they form chemical bonds with free amino-groups. The Edman degradation principle of sequential N-terminal degradation of a polypeptide is entirely based on this. Edman degradation typically comprises two steps, a coupling step and a cleaving step. In general, the coupling step of Edman degradation involves cross-linking ITC (or a suitable analogue thereof) with the amino-group of a peptide's N-terminus at an elevated pH (basic environment), thereby forming an N-terminal thiocarbamyl derivative (in the case of ITC) or the like. Lowering the pH, e.g. by addition of an acid such as trifluoroacetic acid (TFA), results in the cleaving of the ITC-bound N-terminal amino acid from the peptide to form a free anilinothiozolinone (ATZ) derivative or the stable thiohydantoin (TH)-amino acid derivative (in both cases: if ITC was used initially), the latter being then identified with chromatography. By iteratively repeating N-terminal degradation of the peptide, sequence information is generated.

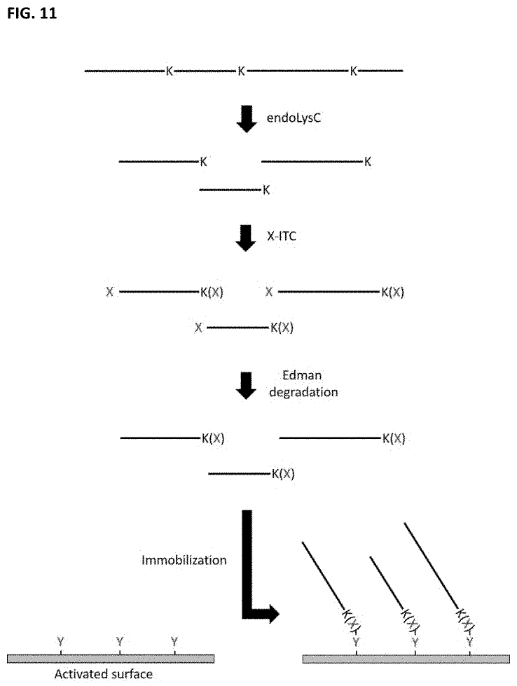

ITC can thus be used to conjugate the N-terminus of a peptide, but not the C-terminus. Here, we describe an elegant way to overcome this problem (FIG. 11). Proteins or polypeptides are first treated with a lysine-specific endoproteinase, such as endoLysC. The result of this step is a mixture of peptides having an amine-group at their N-terminus and a lysine group at their C-terminus. Besides its ability to bind amine-groups, ITC also binds lysine (K) through the primary amine in its side chain. ITC can be coupled by a plethora of (click chemistry) linking partners X, such as DBCO, biotin, . . . Thus by adding ITC-X to the peptide digest, both sides of the peptide will be conjugated to ITC-X. In order to specifically conjugate the C-terminus of the peptides to a surface comprising a (click chemistry) binding partner Y which is compatible and thus able to bind X, the N-terminal bound ITC-X is first removed. This can relatively easy be done by an Edman degradation step (see above). After removing the first N-terminal amino acid of all peptides in the mixture, the peptides within the mixture only contain an ITC-X linker at their C-terminus. In a final stage, a surface comprising a plurality of linker Y is contacted with said mixture to immobilize the peptides from the protein extract to said surface. Said peptides are conjugated through their C-terminus and have a free N-terminus.

An aspect of the application is to provide a method for C-terminal immobilization of one or more peptides on a surface comprising the steps of:
mixing a protein or polypeptide with a lysine-specific endoproteinase to obtain a plurality of peptides;
conjugating an isothiocyanate-linker onto the amine groups of the N-terminus and of the C-terminal lysine side chain of said one or more peptides;
removing the N-terminal amino acid bound to the isothiocyanate-linker of said one or more peptides by applying acidic conditions of pH between 3 and 6 or through a single Edman degradation step or adding trifluoroacetic acid; and
immobilizing the one or more peptides on a surface through the isothiocyanate-linker conjugated to the C-terminal lysine side chain.

In one embodiment, the surface is coated at one or more sides with a molecule Y for binding the immobilization linker. In another embodiment, the surface comprises or is built of nitrocellulose or other membrane materials, polystyrene plates or beads, agarose, beaded polymers, silicon or glass slides. The immobilization is performed under conditions wherein a covalent bond can be made between the peptide and the surface. A covalent bond is resistant to degradation effects when incubated in a Edman degradation reaction solvent.

In a particular embodiment, the linker X and Y which are bound to ITC and the surface respectively are selected from the list consisting of alkyne (e.g. propargyl) and azide (copper(I)-catalyzed alkyn-azide cycloaddition, CuAAC), strained alkyne (dibenzocyclooctyne (DBCO, bicyclononyne (BCN), monofluoro-substituted cyclooctyne (MFCO)) and azide (second generation copper-free click chemistry), trans-cyclooctene (TCO) and tetrazine (third generation copper-free click chemistry), phosphine and azide (Staudinger ligation), sulfhydryl-reactive group (maleimide, haloacetyls (e.g. iodoacetyl, bromoacetyl), pyridyl disulfides) and thiol, and biotin and (strept)avidin.

In another embodiment, said ITC is an isothiocyanate analogue selected from the list consisting of ITC, FITC, CITC, PITC, SPITC, propargyl-ITC and azido-PITC.

In another aspect, a composition is provided comprising a surface, one or more peptides and an immobilization linker, wherein said immobilization linker covalently binds the C-terminus of said one or more peptides to said surface, and wherein said immobilization linker comprises isothiocyanate or an analogue thereof. In one embodiment, the immobilization linker further comprises alkyne (e.g. propargyl), azide (copper(I)-catalyzed alkyn-azide cycloaddition, CuAAC), strained alkyne (dibenzocyclooctyne (DBCO, bicyclononyne (BCN), monofluoro-substituted cyclooctyne (MFCO)), azide (second generation copper-free click chemistry), trans-cyclooctene (TCO), tetrazine (third generation copper-free click chemistry), phosphine, azide (Staudinger ligation), sulfhydryl-reactive group (maleimide, haloacetyls (e.g. iodoacetyl, bromoacetyl), pyridyl disulfides), thiol, biotin and/or (strept)avidin.

In another embodiment, the surface comprises or is built of nitrocellulose or other membrane materials, polystyrene plates or beads, agarose, beaded polymers, silicon or glass slides.

In yet another aspect, a method is provided for producing a surface on which one or more peptides are immobilized through their C-terminus, said method comprises the steps of:
providing a surface comprising Y;
obtaining a plurality of peptides by mixing a protein or polypeptide with a lysine-specific endoproteinase;
conjugating isothiocyanate-X onto the amine groups of the N-terminus and the C-terminal lysine side chain of said plurality of peptides obtained in the previous step, wherein said X is suitable to bind Y;
removing the N-terminal amino acid bound to isothiocyanate-X of said plurality of peptides; and immobilizing the plurality of peptides onto the surface by binding X conjugated to the C-terminal lysine side chain to Y of the substrate.

In one embodiment, the N-terminal amino acid bound to ITC-X is removed by applying acidic conditions of pH between 3 and 6, by adding trifluoroacetic acid or by a single Edman degradation step. In another embodiment, the surface is coated at one or more sides with a molecule Y suitable to bind the immobilization linker. In a particular embodiment, X comprises or consists of an alkyne (e.g. propargyl), a strained alkyne (dibenzocyclooctyne (DBCO, bicyclononyne (BCN), monofluoro-substituted cyclooctyne (MFCO)), trans-cyclooctene (TCO), phosphine, sulfhydryl-reactive group (maleimide, haloacetyls (e.g. iodoacetyl, bromoacetyl), pyridyl disulfides), or biotin and Y comprises or consists of azide (copper(I)-catalyzed alkyn-azide cycloaddition, CuAAC), azide (second generation copper-free click chemistry), tetrazine (third generation copper-free click chemistry), azide (Staudinger ligation), thiol, or (strept(avidin) respectively.

The surface can be made nitrocellulose or other membrane materials, polystyrene plates or beads, agarose, beaded polymers, silicon or glass slides.

The herein disclosed immobilization methods can further comprise one or more drying steps, more particularly between the obtaining and conjugating step, between the conjugating and the removing step and/or between the removing and immobilizing step.

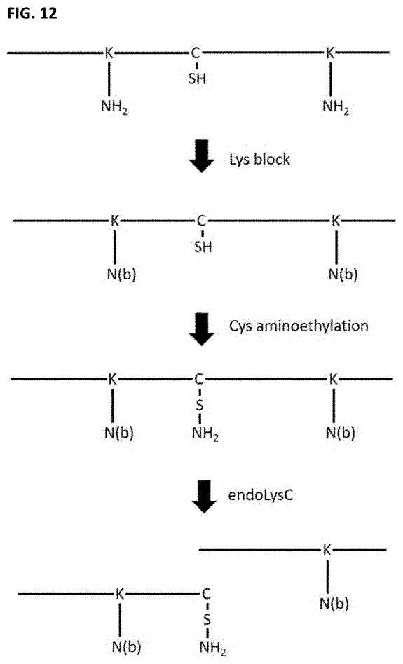

The peptide fragments obtained by an endoLysC digest are typically 10 to 12 amino acids. To obtain longer peptides for single molecule sequencing, lysines can be blocked after which cysteines are converted into S-aminoethyl-cysteines. Considering that lysine-specific endoproteinases can cleave S-aminoethyl-cysteine, and lysines are blocked, proteins will be cleaved after each aminoethylated cysteine (FIG. 12). The same isothiocyanate conjugation method can then be applied on these peptides. Blocking lysines as used herein refers to protecting lysines from enzymatic digest with for example EndoLysC. A number of methods are available to the person skilled in the art to block lysines. Non-limiting examples are acetylation with acetyl N-hydroxysuccinimide ester (acetyl-NHS) and dimethylation with formaldehyde and sodium cyanoborohydride.

Thus the application also provides a method for C-terminal immobilization of one or more peptides on a surface comprising the steps of:
blocking the lysine residues of a protein or polypeptide
aminoethylating one or more cysteine residues to S-aminoethyl-cysteines
mixing the protein or polypeptide with a lysine-specific endoproteinase to obtain a plurality of peptides;
conjugating an isothiocyanate-linker onto the amine groups of the N-terminus and of the C-terminal S-aminoethyl-cysteine side chain of said one or more peptides;
removing the N-terminal amino acid bound to the isothiocyanate-linker of said one or more peptides by applying acidic conditions of pH between 3 and 6 or through a single Edman degradation step; and
immobilizing the one or more peptides on a surface through the isothiocyanate-linker conjugated to the C-terminal lysine side chain.

In one embodiment the blocking step is performed by acetylating the lysine residues with acetyl N-hydroxysuccinimide ester (acetyl-NHS) or by dimethylating them with formaldehyde and sodium cyanoborohydride.

In another embodiment, the aminoethylation step is performed by administering bromoethylamine.

In a particular embodiment, the methods of sequencing a peptide or protein as described herein are provided with an additional immobilization step, more particularly one of the ITC-based immobilisation methods described above.

In various embodiments of current application, the polypeptide may be immobilized on a surface prior to contact with the labeled probe. The peptide may be immobilized on any suitable surface (see later). Crucial for the methods disclosed in current application is that the polypeptide to be sequenced or of which the N-terminal amino acid is to be identified or categorized is immobilized through the moiety which is most C-terminal of the polypeptide or through the moiety C-terminal of the scissile bond. The polypeptide is thus attached to the surface of the application with its C-terminus or with a moiety along the peptide's structure, C-terminal to the scissile bond (e.g. with a cysteine's thiol function through e.g. maleimide chemistry or gold-thiol bonding, well known in the art).

“Scissile bond” as used herein refers to the covalent chemical bond to be cleaved by one or more aminopeptidases.

“Surface” as used herein is a synonym for carrier or layer. The surface, carrier or layer may be nitrocellulose or other membrane materials, polystyrene plates or beads, agarose, beaded polymers, or glass slides. The surface or layer of current application is suitable to use in the detection of molecular labels, electrochemical signals, electromagnetic signals, plasmon related events.

Said molecular label can be an optical (comprising but not limited to luminescent and fluorescent labels) or electrical (comprising but not limited to potentiometric, voltametric, coulometric labels) label.

Said layer can also be a multilayer, i.e. a layer that comprises several layers. In case of a multilayer, at least one layer should allow suitable detection of said molecular labels or said electrochemical, electromagnetic or plasmon related events. Therefore, according to particular embodiments, the surface is an active sensing surface. Hence, the surface immobilized polypeptide of said method of sequencing a surface-immobilized polypeptide at single molecule level is a polypeptide immobilized on an active sensing surface. In more particular embodiments, said active sensing surface is either a gold surface or an amide-, carboxyl-, thiol- or azide-functionalized surface on which the polypeptide of said method is chemically coupled. In other particular embodiments, said carrier is a nanoparticle, a nanodisk, a nanostructure, a chip. In most particular embodiments, said surface is a self-assembled monolayer (SAM).

Silicon as a surface is of particular interest as it is the basis material used in the microelectronic chips and this would enable the transduction of biological and biochemical events more directly to the electronics required to produce practical devices. Silicon also offers compatibility with bulk manufacturing and with developing photonic structures. A further advantage of using a silicon substrate is that it provides an atomically flat surface.

As already discussed herein, the polypeptides immobilized on a surface should be denatured so that the N-terminus is freely accessible (in case the polypeptide is immobilized through its C-terminus) for binding with the probe and for chemical or enzymatic cleavage, but also to avoid steric hindrance or interference of said cleavage. Therefore, the methods of current application are also provided including a first step of polypeptide denaturation. In such denaturing conditions the catalytically active aminopeptidases to be used should withstand the denaturing condition. It is thus preferable that in these cases that the agents used herein (e.g. labeled probes, aminopeptidase, . . . ) are thermophilic and/or solvent resistant.

In various embodiments of this application, the methods herein described for identifying or categorizing N-terminal amino acids from a C-terminally immobilized polypeptide or for obtaining sequence information from said polypeptide are methods executed on a single molecule level.

For single molecule measurements, it is envisaged that polypeptides from the methods of current application are immobilized on an active sensing surface. In particular embodiments, said active sensing surface is either a gold surface or an amide-, carboxyl-, thiol- or azide-functionalized surface on which said polypeptide is chemically coupled.

Detection of Cleavage

One of the additional parts of the methods of the application is that the cleavage of the terminal amino acid is to be detected or confirmed. Hence also provided herein are the methods of current application, additionally including a step of determining the cleavage of said terminal amino acid by measuring an optical, electrical or plasmonical signal of the surface-immobilized polypeptide, wherein a difference in optical, electrical or plasmonical signal is indicative for cleavage of said terminal amino acid. Indeed, immobilized peptides with a free N-terminus have several properties which are utilized to determine when an N-terminal amino acid has been cleaved off.

In a first example, the free N-terminal amine group carries a positive charge under a broad range of pH. The distance between this positive charge and the anchor point of the peptide, through which it is immobilized, can be measured e.g. by measuring the random telegraph noise (Sorgenfrei et al 2011 Nano Lett 11:3739-3743) in potentiometric detection when the peptide is immobilized on a suitably designed detector element (carbon nanotube, nanometer-scale transistor such as field effect transistor, in particular fin-shaped field effect transistors, gate all-around field effect transistors, nanoribbon field effect transistors and the like). Upon cleavage of an N-terminal amino acid, the positively charged N-terminal amino group comes closer to the anchor point of the peptide and thus to the detector surface. In a fully stretched out peptide, the length with which the distance between this charge and the anchor point shortens is about 3.8 angstrom (contour length), as constrained by the geometry of the covalent bonds in the peptide backbone. Hence, under environmental conditions of peptide secondary structure disruption (high temperature, organic (co-)solvent exposure etc.), the maximum of the distribution of length measurements between the amino-terminal charge and the peptide anchor point has an upper limit which is constrained by the geometry of the covalent bonds in the peptide backbone. Measurement of a change in this maximum length by repeated observation of the peptide's amino-terminal charge during the presence of the cleaving-inducing agent reveals the time point at which the cleavage inducing agent has indeed cleaved off an N-terminal amino acid.

In a second example, the amino-terminal amino acid can be reacted with a reagent in such a way that an amino acid derivative is formed in which the positive charge on the terminal amino-group is eliminated, converted to an amino acid derivative carrying one or more negative charges or increased from a single positive charge to a multiply positive charged amino acid derivative. This can be achieved for example with contacting the immobilized peptide with a suitably chosen N-hydroxysuccinimidyl reagent that carries no charge, one or more positive charges or one or more negative charges). Alternatively, the charge-modulating reagent can be the cleavage-inducing reagent itself, as is the case when the immobilized peptide's terminal amino-group is reacted with a suitably chosen isothiocyanate reagent, such as PITC, CITC, SPITC (4-sulfophenylisothiocyanate) or an azidophenyl isothiocyanate, in which the latter can further be modified through click chemistry on the azide group either prior to the contacting of this agent with the immobilized peptide, during or after the contacting of this agent with the immobilized peptide. In this way, the charge difference between the peptide carrying the amino acid derivative and this peptide after the N-terminal amino acid derivative has been cleaved off is rendered binary (conversion of neutral to positive, conversion of negative to positive or conversion of multiple positive charge to single positive charge) or is enhanced, or both.

In a third example, the N-terminal amino acid's amino-group or its side chain can be reacted with an agent that imparts a spectroscopically distinguishable property in such a way that an N-terminal amino acid derivative is generated that can be detected using spectroscopical methods such as fluorimetry, Raman spectroscopy, plasmon resonance etc. In particular, single-molecule detection using total internal reflection fluorescence (TIRF) microscopy is a preferred method, as it is designed to detect fluorescence in a thin layer juxtaposed to the reflective surface, e.g. glass, to which the peptides can be immobilized. Upon contacting the cleavage-inducing agent (which can be e.g. an aminopeptidase, Edmanase or isothiocyanate-containing molecule), the time at which cleavage occurs can be detected by a spectroscopical property change in an observational time series of the immobilized peptide (for example, a loss of fluorescent signal due to cleaving off the fluorescently labeled N-terminal amino acid derivative. Alternatively, a loss of a Förster Resonance Energy Transfer (FRET) signal can be observed when the immobilized peptide contains a suitable FRET donor or acceptor and the N-terminal amino acid derivative contains a matching FRET acceptor or donor. In yet another embodiment, the N-terminal amino acid is derivatized (e.g. with biotin, for example using a biotinylated isothiocyanate) such that a binding agent (e.g. an avidin such as streptavidin or neutravidin) that carries a spectroscopically distinguishable label (e.g. a fluorophore) can bind the derivatized N-terminal amino acid. The time until cleavage of the N-terminal amino acid can then be measured as the time point at which a change occurs in an observational time series of binding competence of the immobilized peptide to said binding agent. Binding competence is the ability of a peptide to bind or not bind to the binding agent, or the characteristics of such binding, such as binding affinity, kₒₙ, k_{off}. Detection can be done using e.g. TIRF.

Kits Comprising 18-Crown-6 Ethers and Uses Thereof

In particular aspects of this application, the use is provided of one or more labeled probes (e.g. crown ether or derivative thereof) of which the kinetics of association with and/or dissociation from an N-terminal amino acid from an immobilized polypeptide is characteristic for and thus identifies said N-terminal amino acid. In one embodiment, the use of a crown ether or derivative thereof to obtain sequence information of a polypeptide immobilized on a surface via its C-terminus or via a peptide moiety C-terminal to the first peptide bond of said polypeptide is provided, wherein the residence time of said crown ether or derivative thereof on the N-terminal amino acid or wherein association and/or dissociation kinetics of said crown ether or derivative thereof on said N-terminal amino acid identifies or categorizes said N-terminal amino acid. In a more particular embodiment, said crown ether is an 18-crown-6 ether or derivative thereof. Even more particularly, said 18-crown-6 ether is a 4′-aminobenzo-18-crown-6 ether, a 4′-aminodibenzo-18-crown-6 ether or a triaza-18-crown-6 ether.

Also the use is provided of one of the herein described crown ethers or derivatives thereof, wherein said crown ethers or derivatives thereof are labeled and/or wherein the label may be partly or wholly integrated in the crown structure.

In yet another aspect of this application, a kit is provided comprising an 18-crown-6 ether or a labeled 18-crown-6 ether and an Edman degradation agent, more particularly ITC or an ITC analogue. In a particular embodiment, the 18-crown-6 ether or the labeled 18-crown-6 ether and said Edman degradation agent are present in a 1:1 ratio, 2:1 ratio, 3:1 ratio, 4:1 ratio, 5:1 ratio or between a 6:1 and a 10:1 ratio, or between a 2:1 and a 20:1 ratio. Also a kit is provided comprising an 18-crown-6 ether or a labeled 18-crown-6 ether and an aminopeptidase. In a particular embodiment, the 18-crown-6 ether or the labeled 18-crown-6 ether and said aminopeptidase are present in a 1:1 ratio, 2:1 ratio, 3:1 ratio, 4:1 ratio, 5:1 ratio or between a 6:1 and a 10:1 ratio, or between a 2:1 and a 20:1 ratio. In particular embodiments, said aminopeptidase is selected from the list consisting of aminopeptidase T from Thermus aquaticus (AMPT_TH EAQ), aminopeptidase T from Thermus thermophilus (AMPT_TH ET8), PepC from Streptococcus thermophiles (PEPC_STRTR), Aminopeptidase S from Streptomyces griseus (APX_STRGG), Aminopeptidase from Streptomyces septatus TH-2 (Q75V72_9 ACTN), Aminopeptidase 2 from Bacillus stearothermophilus (AMP2_GEOSE), the wild-type and engineered Trypanosoma cruzi cruzipain (or cruzain), Thermus aquaticus aminopeptidase T, Streptomyces griseus aminopeptidase (SGAP; UniProtKB-P80561), Aeromonas proteolytica aminopeptidase (APAP; UniProtKB-Q01693), Serratia marcescens aminopeptidase (SMAP; UniProtKB-032449), Pyrococcus furiosus aminopeptidase (PFAP; UniProtKB-P56218), Lactobacillus helveticus X-prolyl dipeptidyl aminopeptidase and Streptomyces griseus X-prolyl dipeptidyl aminopeptidase as depicted in SEQ ID No. 1-14.

In various embodiments, said 18-crown-6 ether as part of said kit is labeled as described in current application. In another embodiment, said 18-crown-6 ether is a Cy5 labelled 4′-aminobenzo-18-crown-6 ether, 4′-aminodibenzo-18-crown-6 ether or triaza-18-crown-6 ether. The kits herein described are also provided for obtaining sequence information of a polypeptide.

The following examples are intended to promote a further understanding of the present invention. While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

### EXAMPLES

### Example 1: TIRF Microscopy for Single Peptide Detection

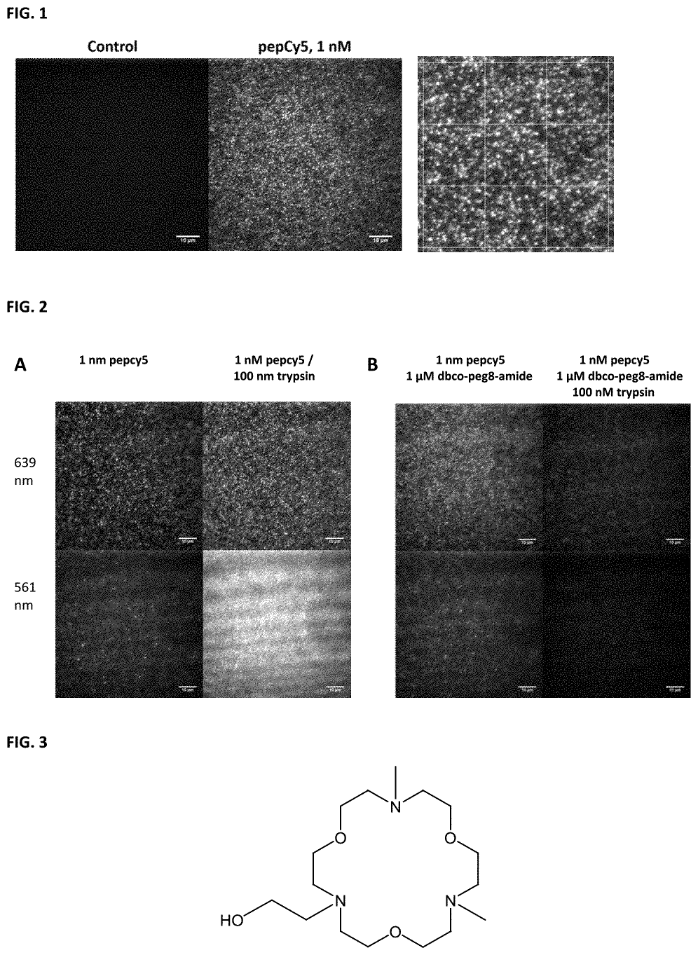

In a first step, a system was developed to immobilize peptides which are to be sequenced on a surface. Azide-functionalized, oven-cleaned glass plates were used as surface and the peptide NNGGNNGGRGNK to which N-terminally a DBCO-PEG8 group and C-terminally a sulfo-Cy5 fluorescent probe was attached was used as test peptide. The test peptide was immobilized through an azide-DBCO click reaction. The azide-functionalized glass plates were placed on top of 1 ml of 1 nM test peptide, and incubated for 24 h in the dark. For functionalization, 11-azido-undecyl(trimethoxy)silane was used which makes the glass surface hydrophobic, allowing the glass to float on the liquid. After 24 h the glass plates were washed 3 times with 1 ml MS grade water (each 30 min washing). For the control sample, glass plates were incubated with water. Microscopy was executed on a Zeiss TIRF microscope and pictures were taken at λ_{Em} 639 nm. The peptides were successfully immobilized on the glass surface, and at a concentration of 1 nM an adequate spatial distribution was obtained (FIG. 1).

### Example 2: Accessibility of N-Terminal Amino Acids of Surface Immobilized Peptides

To check whether N-terminal amino acids of surface immobilized polypeptides are accessible for crown ether, Edman degradation agents and/or aminopeptidase, conditions were optimized using an enzymatic cleavage assay of surface immobilized peptides. The test peptide DBCO-PEG8-NNGGNNGGRGNK-Cy5 was again used but now together with trypsin. Successful enzymatic surface reaction is detected after cleavage at the arginine which removes the fluorescent probe. Azide-functionalized, oven-cleaned glass plates were placed on top of 1 ml of 1 nM test peptide and incubated for 24 h in the dark. After washing 3× with MS grade water, glass plates were incubated for 1 h at room temperature with 100 nM trypsin (sequencing grade, promega). Controls were incubated with water. After trypsin treatment, plates were again washed 3× with water. The experiment was also repeated in the presence of 1 μM of DBCO-PEG8-amide passivator (added to the test peptide during the 24 h azide-DBCO click reaction), to evaluate its effect on clustering and aspecific trypsin surface interaction. The trypsin reaction in absence of passivator was not successful, due to high background signal from aspecific binding of trypsin on the surface (FIG. 2A). Background was assessed in the lower AEm channel of 561 nm (Cy3 channel). When the passivator was present, the background disappeared, and a considerable amount of immobilized peptide was cleaved, as seen by a significant decrease in spots (FIG. 2B).

### Example 3. Transient Associations Between 18-Crown-6 Ethers and N-Termini of Polypeptides

Current application demonstrates proof of concept for the use of 18-crown-6 ethers for the identification of N-terminal amino-acids of surface immobilized proteins. The concept is based on the observation that 18-crown-6 ether derivatives can associate with protonated amines such as amino groups present at the N-termini of peptides or proteins (Yeh et al 2009 J Am Soc Mass Spectrom 20:385-393). To determine the exchange rate of the interaction between N-termini of polypeptides and 18-crown-6 ethers, an NMR study was performed. A model peptide (NH2-Ala-Ala-Phe-amide) was used in combination with the 18-crown-6 analogue triaza-trioxo-crown ether (TTC-ether; FIG. 3). The model peptide was designed with an N-terminal Ala to easily determine the shift of the N-terminal Ala methyl group upon binding to the TTC-ether. Solutions of the peptide were prepared in D2O/D-acetonitrile (3 ml/1 ml) with 10 mM D-acetic acid. A concentration of 100 μM peptide was maintained and mixed with varying concentrations of the TTC-ether. The TTC concentrations ranged from 200 to 150, 100, 75, 50 and 25 μM, thus corresponding with TTC/peptide molar ratios of 2:1, 1.5:1, 1:1, 0.75:1, 0.5:1 and 0.25:1 respectively. The final volumes for NMR analysis were each time 600 μl. Measurements were performed at 20° C.

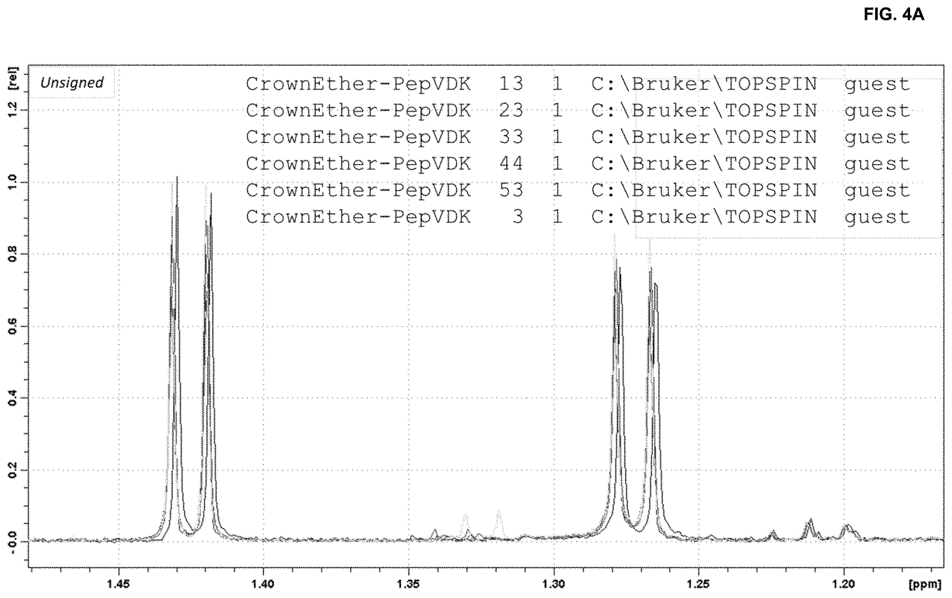

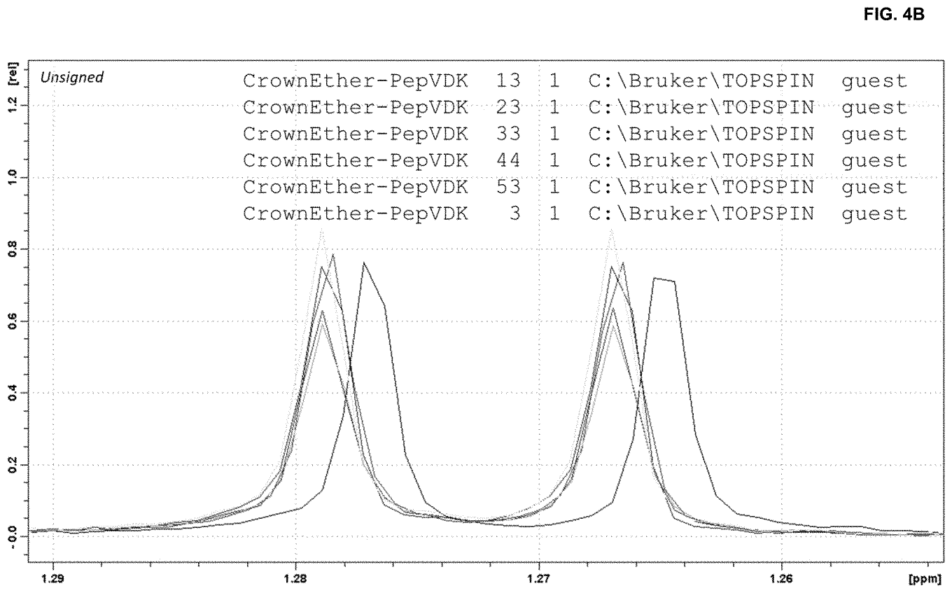

A 2D-TOCSY (Total Correlation Spectroscopy) spectrum allows the identification of the position of the two methyl groups of both Ala residues around 1.25 ppm and 1.43 ppm. Every Ala methyl signal shows up as a doublet in the 1D-NMR spectrum because of the coupling constant to the Ha proton. In order to retrieve information about the binding of the crown ether with the peptide, a titration was performed with different concentrations of the TTC-ether after which the shifts for the Ala methyl groups was measured at high resolution. Indeed, Ala-methyl shifts were observed when different TTC-ether/peptide ratios are measured (FIG. 4A). The shifts are very small, concentration dependent and, interestingly, very comparable for the two Ala-methyl residues. When recording the Ala-methyl doublet in the 1.27 ppm region (the N-terminal Ala) at higher resolution, a clear shift is already noticed when half of the peptide is saturated (FIG. 4B, blue line). There are further small shifts observed upon further increase of the TTC-ether concentration, with no further change once the 1:1 molar ratio is reached (FIG. 4B, red line). The absence of separate signals for free peptide and TTC-peptide complex suggests stoichiometric binding, with the free Ala resonance coexisting with the bound Ala resonance. This is indicative for a fast exchange rate, which can be estimated to be faster than one exchange per second (NMR time±1 sec).

The same experiments were repeated with the acetylated peptide Acetyl-Ala-Ala-Phe-amide. As expected we did not measure any noticeable interaction.

### Example 4. Fluorescent Labeling of Crown Ethers

In order to monitor the residence time of the crown ether on the N-terminal amine of immobilized peptides with total internal reflection fluorescence (TIRF) microscopy, a fluorescently labeled crown ether has to be obtained. For this, Cy5-NHS ester was coupled on the free amine of the crown ethers 4′-aminobenzo-18-crown-6 (FIG. 5A) and 4′-aminodibenzo-18-crown-6 (FIG. 5B). A total of 200 nmol of crown ether was mixed with 5000 nmol Cy5-NHS ester (25× molar excess) in 4:4:2 pyridine/acetonitrile/water (final volume of 100 μl, and incubated overnight at room temperature. The mixture was then dried and dissolved in 0.1% TFA in 25% ACN.

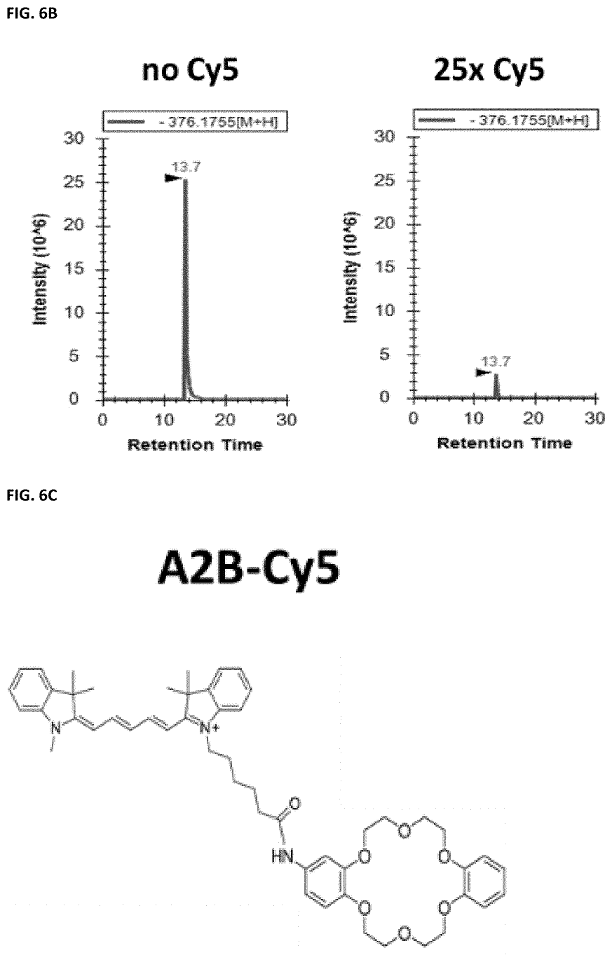

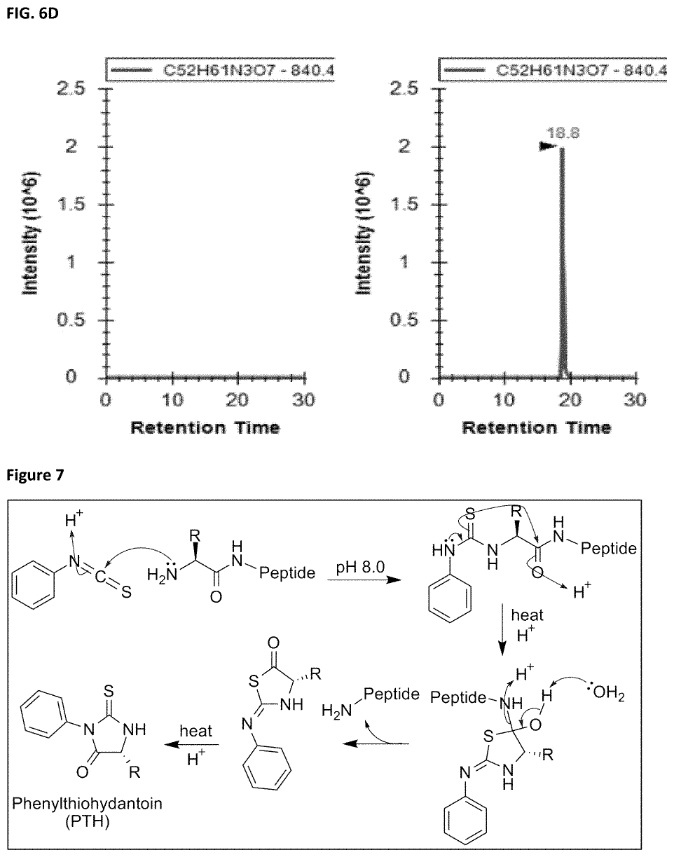

As can be seen in FIG. 6, conjugation of 4′-aminodibenzo-18-crown-6 (A2B) with Cy5 could be achieved. Conjugation worked equally well for 4′-aminobenzo-18-crown-6 (data not shown). Based on the decrease of unconjugated A2B, the conjugation efficiency was estimated to be ±90% (FIG. 6). The fluorescent crown ether can also be purified with RPLC because the Cy5-conjugated crown ether is even more hydrophobic than either the free crown ether and free Cy5.

Based on the fluorescence, the binding of the crown ether on the immobilized polypeptide can be detected. The residence time of the crown ether on the N-terminal amino acid of said immobilized polypeptide can be measured and correlated with the nature of the bound N-terminal amino acid. The further interesting aspect of this interaction is that it can be easily stopped at high pH; indeed this interaction is only taking place with the protonated form of the amino-group. The interaction is therefore especially useful in combination with the Edman degradation chemistry.

Thus successive treatment of the protein N-termini exposed after each Edman degradation cycle with Cy5-18C6 will allow the reading of the amino acid sequence based on the association and/or dissociation kinetics and/or the residence time of the labeled crown ether on the N-terminal amino acid.

### Example 5. Crown Ether Binding Study Using Peptide Arrays

To study the binding of crown ethers to different peptides, a peptide array is used (PEPperPRINT array), containing an array of 400 peptides comprising peptides with all amino acid combinations at the first and second N-terminal position ([AA1][AA2]GGNNGG; four replicates of each peptide). After applying a fluorescently labeled crown ether (Cy5.5-dibenzo-18-crown-6), the relative affinity for all peptide substrates is determined by the fluorescence intensity on each peptide spot. The crown ether was applied on the peptide array at a concentration of 0.1 μg/μl in 0.1% triethanolamine in 50% ACN. After binding the array was washed two times for 10 sec using the same buffer. Finally, the fluorescence intensity was measured using a LI-COR Odyssey Imaging system. An image of the array fluorescence after binding clearly shows differential binding, dependent on the first but also second amino acid of the peptide (FIG. 10, A). When comparing the fluorescence intensity of the peptides with variable N-terminal amino acid, but all with leucine at the second position, binding appears to be strongest on N-terminal glycine (due to minimal steric hindrance) (FIG. 10, B). The binding is weakest on N-terminal proline. When the binding is compared on peptides with N-terminal glycine but with variable second amino acid, the binding is strongest on the peptides GFGGNNGG, GLGGNNGG, GYGGNNGG, GIGGNNGG, GKGGNNGG and GWGGNNGG. The large hydrophobic side chains of the second amino acid likely interact with the hydrophobic regions of the crown ether (crown ether benzene rings and/or cy5.5 fluorophore). These results clearly demonstrate that crown ether binding can distinguish immobilized peptides from each other.

### Example 6. Monitoring Crown Ether Binding Kinetics at the Single Molecule Level

To monitor the true single molecule binding kinetics of the fluorescently labeled 18-crown-6 ethers, a set of 20 peptides, containing all N-terminal amino acid combinations at the first N-terminal position ([AA1]GGNNGG), is each immobilized onto a glass surface through their C-termini after which the crown ether residence time on the N-termini is recorded with TIRF microscopy. For each N-terminal amino acid, the crown ether “on-time” or “residence time” is determined during a predetermined time period. From the obtained “on-time” data, the different amino acids are divided in categories in order to identify amino acid categories in polypeptides with unknown sequence.

In a next step, the peptides are subjected to one or more degradation cycli, either by Edman degradation or by aminopeptidases, to determine the crown ether “on-time” on the next amino acid(s).

### Example 7. The C-Terminal Conjugation of the Peptide GAGSSEPVTGLDAK with Propargyl-Isothiocyanate

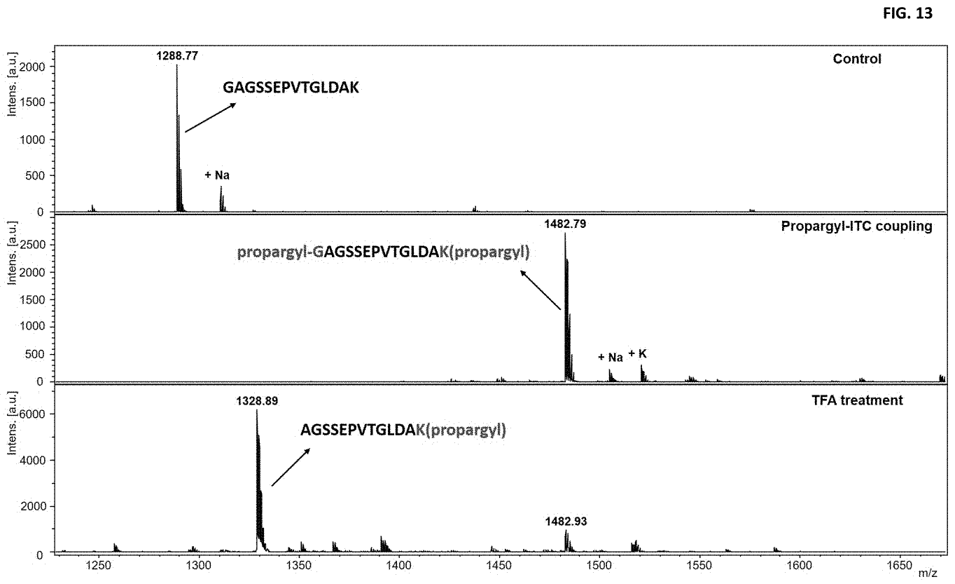

Propargyl-isothiocyanate is first conjugated to both the N-terminal amine and the amine of the C-terminal lysine side chain. After one Edman degradation step only the C-terminal lysine side chain remains modified, while the N-terminal modification is removed together with the N-terminal amino acid (FIG. 13).

### Example 8. The Conversion of Cysteine to S-Aminoethyl-Cysteine with Bromoethylamine, and Subsequent Cleavage with LysC Endoproteinase

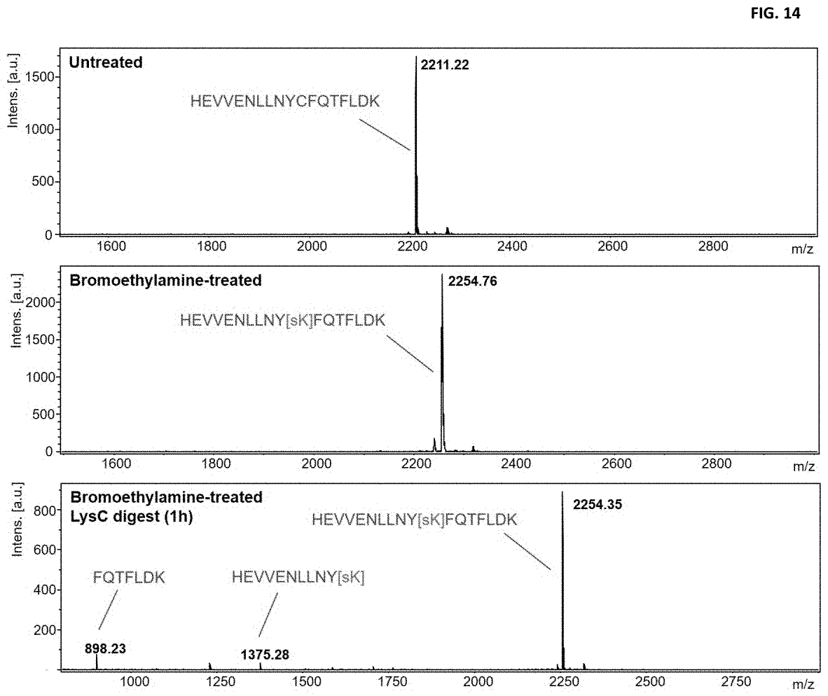

First, the cysteine in HEVVENLLNYCFQTFLDK was aminoethylated to S-aminoethyl-cysteine using bromoethylamine. Then a LysC digest was performed for 1 h. The peptide was cleaved at the S-aminoethyl-cysteine (FIG. 14).

### Example 9. The ITC-Based C-Terminal Conjugation of the Peptide GAGSSEPVTGLDAK with Biotin

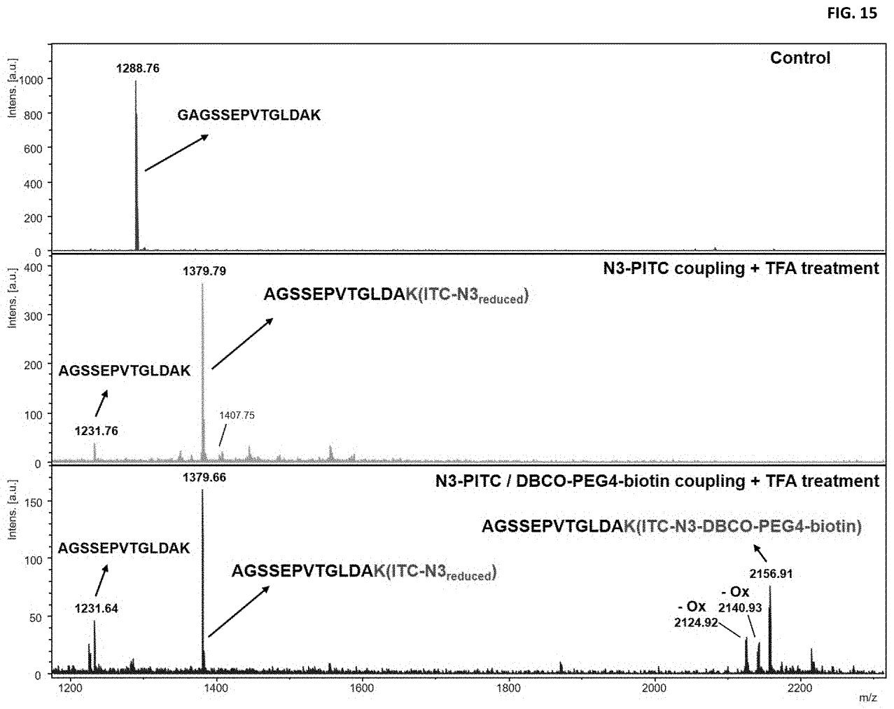

Azidophenyl isothiocyanate (N3PITC) is first conjugated to both the N-terminal amine and the amine of the C-terminal lysine side chain. At the same time, DBCO-PEG4-biotin is clicked on the azide moiety of N3PITC. After one Edman degradation step, only the C-terminal lysine side chain remains modified, while the N-terminal modification is removed together with the N-terminal amino acid (FIG. 15). Any free azide is reduced to an amine during the process.

### Example 10. Immobilization and Single Molecule Detection of Propargyl-ITC Conjugated Peptide

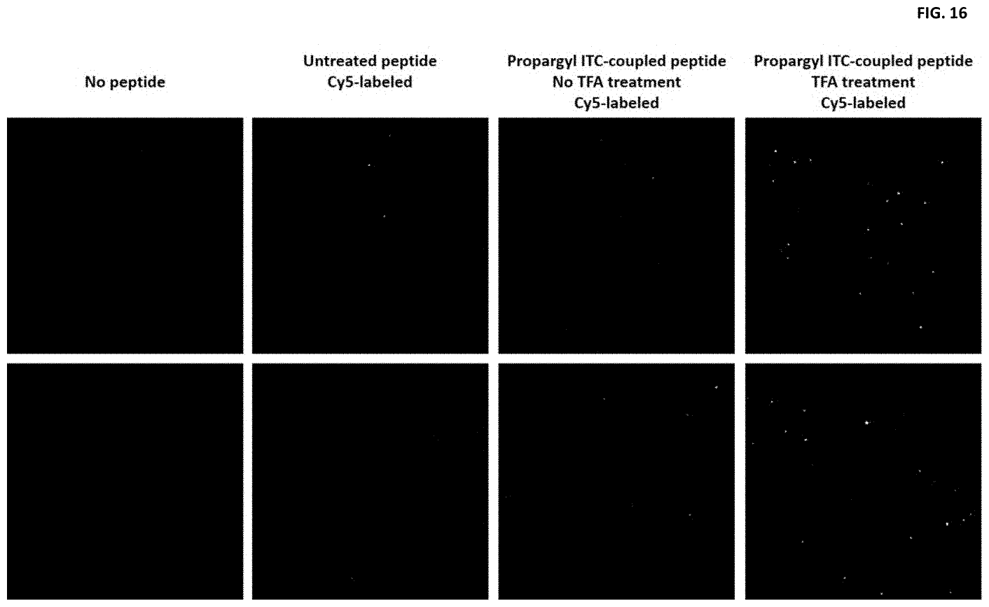

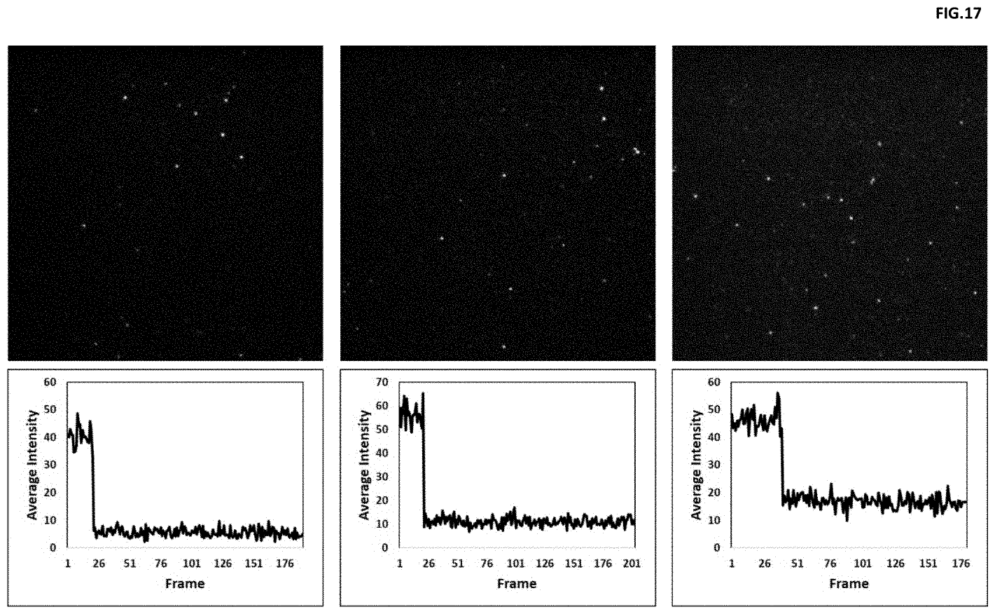

The peptide GAGSSEPVTGLDAK was first conjugated with propargyl-isothiocyanate at the N-terminal amine and C-terminal lysine side chain amine. After treatment with TFA, the N-terminal propargyl-ITC group is removed (together with the N-terminal glycine), while the C-terminal propargyl-ITC group is unaffected. The freed N-terminus was then labeled with sulfo-Cy5-NHS, and by using CuAAC the peptide was immobilized on an azide surface. Single molecule detection of immobilized peptides was performed with total internal reflection fluorescence (TIRF) microscopy (FIG. 16). The detection of single molecules was verified by looking at the individual bleaching curves of each signal. A single, discrete drop in signal intensity (bleaching of Cy5) is indicative for single molecule detection (FIG. 17).

### Experimental Procedure

Isothiocyanate-based C-terminal conjugation of LysC endoproteinase peptides Dried peptide (50 nmol) was resuspended in 16 μl ethanol, after which 32 μl of X-ITC (50 nmol/μl in DMSO or ACN) and 32 μl pyridine was added. The mixture was incubated for 2 h at room temperature. After drying the mixture, 50 μl of TFA was added and the Edman degradation reaction was proceeded for 1 h at room temperature.

Conversion of cysteines to S-aminoethyl-cysteines Dried peptide (50 nmol) was resuspended in 100 μl 100 mM Tris, after which 10 μl of 100 mM TCEP was added. The mixture was incubated for 1 h at 70° C. while shaking, and then cooled back to room temperature. Then 80 μl of 50 mM bromoethylamine (in 100 mM Tris) was added, and incubated at room temperature for 4 h.

Isothiocyanate-Based C-Terminal Biotin Conjugation of LysC Endoproteinase Peptides

Dried peptide (50 nmol) was resuspended in 25 μl ethanol, after which 50 μl of a mixture of 50 nmol/μl N3-PITC and 50 nmol/μ1 DBCO-PEG4-biotin in DMF was added, followed by 50 μl pyridine. The mixture was incubated for 2 h at room temperature. After drying the mixture, 100 μl of TFA was added and the Edman degradation reaction was proceeded for 1 h at room temperature.

Sulfo-Cy5 Labeling of Peptides

Dried peptide (50 nmol) was resuspended in 100 μl mM sulfo-Cy5-NHS in pyridine/acetonitrile/water (2:2:1), and incubated for 2 h at room temperature pyridine/acetonitrile/water (2:2:1), and incubated for 2 h at room temperature.

Copper-Catalyzed Alkyn-Azide Cycloaddition (CuAAC)

Dried, propargyl-ITC conjugated peptide was resuspended in sodium ascorbate/Na₂HPO₄/citric acid buffer (aqueous acidic buffer (pH 5) with sodium ascorbate (125 mM), Na2HPO4 (493 mM), and citric acid (254 mM)) to a concentration of 2 nM. Then 50 μl was taken and mixed with 37.5 μl DMSO and 12.5 of freshly prepared aqueous solution of CuSO₄/Tris(3-hydroxypropyltriazolylmethyl-amine (THPTA) (20 mM CuSO₄ and 20 mM THPTA in water). The mixture was added to Ibidi TIRF μ-Slide VI 0.5 glass bottom flow chambers, of which the surface was previously functionalized with azide, and incubated overnight at room temperature. Finally, the flow chambers were washed three times with water and three times with 50% acetonitrile

Azide-Functionalization of TIRF Flow Cell Glass Surface

After washing Ibidi TIRF μ-Slide VI 0.5 glass bottom flow chambers with acetone, a 5% solution of (3-mercaptopropyl)trimethoxysilane in acetone was added. After 5 minutes of reaction, the flow chambers were washed once with acetone, and then three times with water. The obtained thiol-surface was then converted to an azide surface through the addition of 5 mM bromoacetamido-PEG3-azide in 100 mM Tris. After 2 h incubation, the flow chambers were washed three times with 50% acetonitrile and three times with water.

TIRF Microscopy

Immobilized peptides were visualized with a Zeiss TIRF Observer z.1 microscope, equipped a 100× PlanApo objective (NA 1,46) and with two EMCCD Hamamatsu cameras (exposure time 25 msec, EM gain 100). A 639 nm laser was used for Cy5 detection, operated at 100% laser power. For time series recordings, Definite Focus control was used (focusses every 5 time points).

1. A method of obtaining sequence information of a polypeptide, the method comprising:
associating the N-terminal amino acid of the polypeptide with a crown ether or a derivative thereof, and
measuring the residence time of the crown ether or derivative thereof on the N-terminal amino acid of the polypeptide.

2. The method according to claim 1, wherein the polypeptide is immobilized on a surface via its C-terminus and wherein the residence time identifies or categorizes the N-terminal amino acid.

3. The method according to claim 1, wherein the crown ether is an 18-crown-6 ether or derivative thereof.

4. The method according to claim 1, wherein the crown ether or derivative thereof is labeled.

5. A method for sequencing a polypeptide molecule immobilized on a surface via its C-terminus, the method comprising:
a. contacting the surface immobilized polypeptide with a labeled probe, wherein the probe associates with the N-terminal amino acid of the polypeptide;
b. measuring the association and/or dissociation kinetics of the probe on the N-terminal amino acid;
c. comparing the association and/or dissociation kinetics to a set of reference values characteristic for said probe and a set of N-terminal amino acids, thereby identifying the N-terminal amino acid of the immobilized polypeptide; and
cleaving the N-terminal amino acid of the polypeptide

6. The method according to claim 5, wherein the probe is a crown ether or derivative thereof.

7. The method according to claim 6, wherein the crown ether is an 18-crown-6 ether or derivative thereof.

8. The method according to claim 5, wherein the N-terminal amino acid of the immobilized polypeptide is cleaved by isothiocyanate or an isothiocyanate analogue or by an aminopeptidase.

9. The method according to claim 5, wherein the association and/or dissociation kinetics are measured optically, electrically or plasmonically.

10. The method according to claim 5, additionally comprising determining the removal of the N-terminal amino acid by measuring a signal from the surface-immobilized polypeptide.

11. A kit comprising:
a labeled 18-crown-6 ether or derivative thereof; and
an Edman degradation agent and/or an aminopeptidase.

12. (canceled)

13. The use method according to claim 3, wherein the 18-crown-6 ether is selected from the list consisting of 4′ aminobenzo-18-crown-6 ether, 4′ aminodibenzo-18-crown-6 ether and triaza-18-crown-6 ether.

14. The method according to claim 7, wherein the 18-crown-6 ether is selected from the list consisting of 4′ aminobenzo-18-crown-6 ether, 4′ aminodibenzo-18-crown-6 ether, and triaza-18-crown-6 ether.

15. The kit of claim 11, wherein the 18-crown-6 ether is selected from the list consisting of 4′ aminobenzo-18-crown-6 ether, 4′ aminodibenzo-18-crown-6 ether, and triaza-18-crown-6 ether.

16. The method according to claim 5, further comprising repeating the contacting, measuring, comparing, and cleaving at least once.
